(19) United States
(12) Patent Application Publication
(10) Pub. No.: US 2024/0424373 A1
(43) Pub. Date: Dec. 26, 2024

(54) IMPACT APPARATUS WITH REAL-TIME FEEDBACK

(71) Applicants: Aaron Jake Merrell, Orem, UT (US); Trevor Emil Christensen, Alpine, UT (US); Jake Duane Sundet, Mapleton, UT (US); Ian Matthew Jensen, Provo, UT (US); Maxwell Tree, Provo, UT (US)

(72) Inventors: Aaron Jake Merrell, Orem, UT (US); Trevor Emil Christensen, Alpine, UT (US); Jake Duane Sundet, Mapleton, UT (US); Ian Matthew Jensen, Provo, UT (US); Maxwell Tree, Provo, UT (US)

(73) Assignee: Nano Composite Products, Inc., Orem, UT (US)

(21) Appl. No.: 18/698,619

(22) PCT Filed: Oct. 5, 2022
(86) PCT No.: PCT/US2022/077629

§ 371 Date: Apr. 4, 2024

Related U.S. Application Data
(60) Provisional application No. 63/262,121, filed on Oct. 5, 2021.
(60) Provisional application No. 63/266,805, filed on Jan. 14, 2022.

(51) Int. Cl. A63B 69/32; A63B 24/00; A63B 71/06

(52) U.S. Cl. CPC A63B 69/322; A63B 24/0062; A63B 71/0669; A63B 2220/17; A63B 2220/53; A63B 2220/58; A63B 2220/62; A63B 2244/10

(57) ABSTRACT

An impact apparatus and a computing system provide real-time feedback about a particular activity performed using the impact apparatus. In one general aspect, a method includes providing a user interface that displays a plurality of impact zones, the plurality of impact zones corresponding to a plurality of impact zones on an impact apparatus configured to generate voltage in response to an impact. Implementations can include determining a hit impact location and a velocity of an object for an impact event and updating the user interface with the hit location and velocity. Implementations can include determining a response time for an impact event. Implementations can include determining a location and magnitude of an impact event. Some implementations may include determining whether a location is a target location. Implementations can score an impact according to its magnitude, location, and/or response time.

### CROSS REFERENCE TO RELATED APPLICATIONS

This application is a non-provisional of, and claims priority to, U.S. Provisional Application No. 63/262, 121, filed Oct. 5, 2021, titled “Smart Sports Targets” and to U.S. Provisional Application No. 63/266,805, filed on Jan. 14, 2022, titled “Smart Sports Targets,” which are incorporated by reference herein in their entirety.

### TECHNICAL FIELD

This description relates to an apparatus with a plurality of impact zones and to methods and systems for analyzing and providing activity-specific feedback for impact events detected using the apparatus.

### BACKGROUND

Impact apparatuses, e.g., targets, bags, sleds, pads, etc., are used to improve specific skills in various activities. For example, a pitching target can be used to improve pitching, a bag can be used in martial arts and boxing to practice kick and/or punching form as well as combinations of these strikes, a padded sled can be used in football to practice tackles, etc. These impact apparatuses do not themselves provide any type of feedback but serve as the recipient of an impact event.

### SUMMARY

Disclosed implementations relate to systems that include an improved impact apparatus and a computing system in communication with the impact apparatus to provide real-time feedback about the particular activity performed using the impact apparatus. Put another way, systems and methods are disclosed that can receive, analyze, and provide feedback on impact location and magnitude in multiple configurations. Disclosed implementations can use a sensor in an impact zone that generates a voltage (generates electric potential) when impacted. The voltage is generated without a current producing device. The sensor generates a voltage that is proportional to the magnitude of impact (impact energy). The impact apparatus and/or the computing system is configured to determine a location of the impact on the impact apparatus. In response to an impact event, the system detects, records, and analyzes the voltage response recorded by the impact apparatus to determine impact location(s). In some implementations, the impact location (e.g., hit impact zone) is determined by the impact zone that measures the largest voltage response. In some implementations, any impact zone measuring a voltage response is considered a hit impact zone. In some implementations, the system may determine the impact magnitude. The impact magnitude is determined by an impact analysis application. The impact analysis application is configured to evaluate many different characteristics of the voltage response, such as integrals, maximums, and minimums of the voltage over time. The evaluation performed by the impact analysis application can be activity specific. The impact analysis application can include user interfaces that enable a user to select target impact zones, assign different points/weights to different impact zones, communicate a desired impact sequence to a user, and provide feedback on a sequence of impacts, as described herein.

The details of one or more implementations are set forth in the accompanying drawings and the description below. Other features will be apparent from the description and drawings, and from the claims.

### BRIEF DESCRIPTION OF THE DRAWINGS

FIG. 1 is a high-level block diagram that illustrates an example of a system that includes an impact apparatus and a computing device configured to provide user interfaces that interact with the impact apparatus, according to an implementation.

FIG. 2 is a schematic diagram of an example impact zone of an impact apparatus, according to an implementation.

FIG. 3 is a schematic diagram of an example impact apparatus with a plurality of impact zones, according to an implementation.

FIGS. 4A and 4B illustrate example user interfaces that interact with the impact apparatus of FIG. 3, according to an implementation.

FIG. 5 is a flowchart that illustrates an example process for determining velocity of an object striking an impact zone of an impact apparatus, according to an implementation.

FIG. 6A is a schematic diagram of an example impact apparatus with a plurality of impact zones, according to an implementation.

FIG. 6B is an illustration of the impact apparatus of FIG. 6A affixed to a punching bag, according to an implementation.

FIG. 7 is flowchart that illustrates an example process for scoring impacts to an impact apparatus, according to an implementation.

FIG. 8 illustrates an example user interface, according to an implementation.

FIG. 9 is a flowchart that illustrates an example process for scoring impacts to an impact apparatus based on an impact profile, according to an implementation.

FIG. 10 is a flowchart that illustrates an example process for scoring repeated impacts to an impact apparatus, according to an implementation.

### DETAILED DESCRIPTION

Disclosed implementations relate to analyzing and providing activity-specific analysis of impact events detected by an apparatus with a plurality of impact zones. Disclosed implementations include an impact apparatus and an impact analysis application in communication with each other. In some implementations, a user can use an interface generated by the impact analysis application to select one or more target impact zones. The user interface may be configured to provide feedback on an impact to an impact apparatus, e.g., which impact zone or zones generated a voltage, the magnitude of the impact, etc. The user interface may be configured to record session data and/or provide a history analysis of impact events.

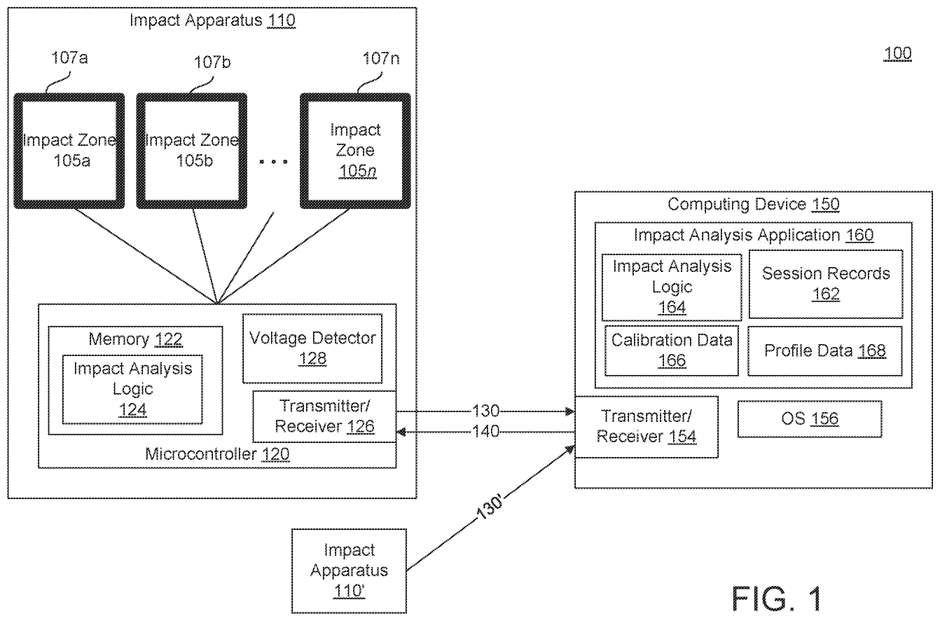

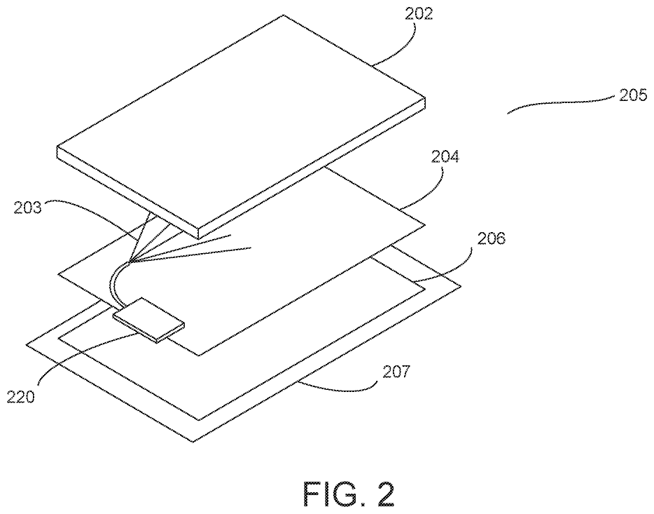

FIG. 1 is a high-level block diagram that illustrates an example of a system 100 that includes an impact apparatus 110 and a computing device 150 configured to provide user interfaces that interact with the impact apparatus 110, according to an implementation. The system 100 may include impact apparatus 110. Impact apparatus 110 may include a plurality of impact zones 105. Each impact zone 105 (e.g., impact zone 105a, 105b, . . . , 105n) defines an area of the impact apparatus 110. Each impact zone 105 can comprise a sensor that generates a voltage (generates electric potential) when deformed, the voltage being generated without a current producing device (e.g., a battery). The sensor may be a polymeric foam with conductive elements disposed on or in the polymeric foam. The sensor can be a foam sensor described in the disclosure of U.S. Pat. No. 10,260,968 or U.S. Pat. No. 8,984,954, the disclosures of which are incorporated by reference. In some implementations, each impact zone 105 may comprise a separate sensor that generates a voltage response proportional to impact energy when impacted (deformed). In some implementations, each impact zone 105 may be defined by the location of conductive electrodes on a single sensor. An example impact zone is also described in more detail with regard to FIG. 2.

One or more impact zone 105 may have a feedback device 107 associated with the impact zone 105. The feedback device 107 may be a device that changes appearance, such as an LED light strip, haptic feedback in the form of vibration on the feedback device 107 or the computing device 150, or a sound. In some implementations, the feedback device 107 surrounds its impact zone 105. In some implementations, the feedback device 107 provides a background appearance to the impact zone 105. In some implementations, the feedback device 107 may be or may further include a device that provides a sound (e.g., plays a .wav. .mp3, etc., file) when the impact zone 105 experiences an impact event or when an exercise routine begins and ends. In some implementations, each impact zone 105a, 105b, . . . , 105n may have a respective feedback device 107a, 107b, . . . , 107n. In some implementations, not every impact zone 105 has a feedback device 107a. For example, in some implementations, the system 100 may include an extension to the impact apparatus 110, e.g., impact apparatus 110′. Impact apparatus 110′ may include all the elements of impact apparatus 110, including the microcontroller and the associated elements illustrated in microcontroller 120, in addition to one or more impact zones 105. In some implementations, one or more impact zones 105 of the impact apparatus 110′ may lack an associated feedback device 107. For example, in a system 100 designed for American football, the impact apparatus 110 may be a football sled and the impact apparatus 110′ may be padding worn by a player. In another example, the impact apparatus 110′ may be a soccer ball and the impact apparatus 110 a target at which the soccer ball is kicked. Thus, impact apparatus 110′ is an example of a second impact apparatus 110 used by the system 100. Accordingly, both impact apparatus 110 and impact apparatus 110′ communicate with the computing device 150 and can provide voltage information 130′ to the computing device 150.

Each impact zone 105 of the impact apparatus 110 generates a voltage in response to deformation. The voltage generated has a direct correlation to the magnitude of the deformation, i.e., the energy of the impact event and is repeatable over time without significant drift. The impact apparatus 110 includes a microcontroller 120. The microcontroller 120 is configured with a voltage detector 128. The voltage detector 128 is operably coupled to the impact zones 105, e.g., via wires connected to electrodes in contact with (disposed on, adhered to, disposed in) an area of polymeric foam for the impact zone 105. The impact apparatus 110 may include a voltage detector 128 operatively coupled to the impact zones 105. In some implementations the microcontroller 120 may include a plurality of voltage detectors 128, each operatively coupled to an impact zone 105. The voltage detector 128 may be capable of detecting voltage generated by the impact zones 105 when the impact zones 105 experience deformation, for example due to an impact. The voltage detector 128 may be any device that detects voltage or produces a value representing the voltage that can be stored, e.g., in memory 122. In some implementations (not shown), the voltage detector 128 may be separate from, but in communication with the microcontroller 120.

The impact apparatus 110 can include microcontroller 120. The microcontroller 120 may be a wireless micro-controller. Non-limiting examples of the microcontroller 120 include the Adafruit Feather and the NRF452840. The microcontroller 120 may enable the impact apparatus 110 to have a small form-factor while still being able to transmit voltage data to computing device 150, which has greater capacity to analyze the voltage data. The small form factor of the voltage detector 128, the memory 122, and the transmitter/receiver 126 allow existing products to be fitted with/designed as the impact apparatus 110 without significant redesign. The small form-factor also results in an impact apparatus 110 that is highly portable.

The microcontroller 120 may be operatively coupled to (include) memory 122 and/or transmitter/receiver 126. The memory 122 may be any type of volatile or non-volatile memory capable of storing data. In some implementations, the microcontroller 120 may be capable of converting detected voltage into a value that is stored in the memory 122. In some implementations the microcontroller 120 is configured to associate detected voltage and/or the value representing detected voltage with the impact zone 105 that generated the voltage. Thus, in some implementations, memory 122 may store voltage data by impact zone. In some implementations, the memory 122 may store additional information with the voltage value, such as the date and/or time the value was detected. The memory 122 may also store other information with the voltage value. The voltage value(s) for the impact zone(s) and additional information, if any, are considered voltage data. Thus, the memory 122 may store voltage data detected after an impact event. In some implementations, the memory 122 may store voltage data for two or more impact events, e.g., a series of impact events. The memory 122 may store the voltage data for impact events until the voltage data is transmitted to a computing device 150, e.g., either wirelessly or via a wired connection.

In some implementations, the microcontroller 120 includes transmitter/receiver 126. The memory 122 may thus be operatively coupled to a transmitter/receiver 126. The transmitter/receiver 126 may be capable of transmitting and/or receiving data wirelessly, e.g., via short-range communications such as BLUETOOTH, Zigbee, Z-Wave, 6LoWPAN, or WiFi. It also may be transmitted via long-range wireless networks such as LTE or 5G. The transmitter/receiver 126 may be capable of transmitting data via a wired connection, such as a Universal Serial Bus (USB) cable. In some implementations, the transmitter/receiver 126 may transmit the voltage data 130 from the memory in response to a command from a computing device, such as computing device 150. In some implementations, the transmitter/receiver 126 may be configured to transmit the voltage data 130 in response to the data being stored in the memory 122. In some implementations, the microcontroller 120 may include logic that formats the voltage data 130 (e.g., associates an impact zone 105 with voltages measured during an impact event at the impact zone 105) and causes the transmitter/receiver 126 to transmit the voltage data 130.

In some implementations, the microcontroller 120 includes impact analysis logic 124. The impact analysis logic 124 may be code, e.g., stored in memory 122, configured to determine if an impact has occurred, e.g., by setting a flag when a voltage threshold is reached on any impact zone. In some implementations, the impact analysis logic 124 may determine the magnitude of the impact, which directly correlates to the impact energy, based on voltage data sampled from the impact zones 105. In such implementations, this magnitude may be included in the voltage data 130 sent to the computing device 150. In some implementations, this calculation may be performed by impact analysis logic 164, e.g., running on computing device 150.

The system 100 includes computing device 150. The transmitter/receiver 126 may transmit voltage data 130 to the computing device 150. In some implementations, the computing device 150 is an external computing device, separate from the impact apparatus 110. The computing device 150 may include a transmitter/receiver 154. The transmitter/receiver 154 is any device configured to operably communicate with the transmitter/receiver 126. In some implementations (not shown), the computing device 150 may be incorporated into the impact apparatus 110. The computing device 150 may be any type of computing device, a tablet, a laptop, a smartphone, a netbook, a desktop, a server, a screen with a processor, a wearable (watch, fitness tracker, glasses), etc. The computing device 150 may include an impact analysis application 160. The impact analysis application 160 can be a native application, a web application, a progressive web application, or any type of application compatible with the operating system 156 of the computing device 150.

The impact analysis application 160 may be configured to provide a user interface (or various user interfaces) for interacting with the impact apparatus 110. For example, the impact analysis application 160 may be configured to provide a user interface that includes a plurality of virtual impact zones. A virtual impact zone is a graphical representation of one of the impact zones 105. A virtual impact zone thus corresponds to, and is a representation of, an impact zone 105 of the impact apparatus 110, i.e., a physical impact zone. As used herein, an impact zone can refer to a virtual impact zone and/or its corresponding physical impact zone. When used in the context of a user interface, such as one generated by impact analysis application 160, the impact zone is a virtual impact zone. When used in the context of an impact apparatus, such as impact apparatus 110, the impact zone is a physical impact zone. Each virtual impact zone corresponds to one physical impact zone, thus reference to “an impact zone” can refer to a virtual impact zone, a physical impact zone, or both a physical impact zone and its virtual representation.

The impact analysis application 160 may be configured to analyze the voltage data 130 received from the impact apparatus 110, e.g., using impact analysis logic 164. Analysis of the voltage data may include determining impact energy of an impact event. Analysis of the voltage data may include determining a location (or locations) of the impact event. Analysis of the voltage data may include using the determined impact energy and/or locations to calculate a score for the impact event. Where the voltage data 130 includes voltage data for a series of impact events, analysis of the voltage data 130 can include analysis of the series. Some of the analysis can be performed in conjunction with impact analysis logic 124, which may provide the result of the analysis as part of voltage data 130. Analysis of the voltage data is described in more detail herein with respect to the different activities performed in conjunction with the impact apparatus 110.

In some implementations, the impact analysis application 160 may have access to calibration data 166. The calibration data 166 may enable the impact analysis application 160 to convert the voltage data into impact energy, impact force, peak force, impact velocity, impact mass, etc. The impact zones 105 are configured to generate a voltage when impacted that is proportional to the magnitude of impact. For example, the impact zones 105 may be a polymeric foam with conductive fillers. The composition of the foam (e.g., amount/type of conductive fillers, foam base used, method of curing the foam, etc.) affect the proportion. The calibration data 166 includes data representing the proportion (e.g., determined by controlled impact events where the impact energy is known and recording the voltage responses). In other words, the calibration data 166 includes data that enables the impact analysis application 160 to convert the voltage data to impact energy, impact force, peak force, impact velocity, impact mass, etc. In some implementations the calibration data 166 may be provided to the computing device 150. In some implementations, the computing device 150 may include a module (not shown) that collects and stores the calibration data 166. An impact apparatus 110 that is manufactured outside of a controlled environment (e.g., outside of an established manufacturing process) may need to be calibrated after each manufacture. An impact apparatus 110 that is manufactured in a controlled environment, however, may not need calibration after every manufacture.

In some implementations, impact zones 105 may include zones with different foam properties. In such implementations, the calibration data 166 may include calibration data for specific impact zones. In some implementations, the calibration data 166 may also include information about objects used to impact the impact apparatus 110. The information about an object can include its mass. Thus, a user may be able to select an object of known mass, e.g., using a user interface generated by the impact analysis application 160. Using the known mass, the impact analysis application 160 can calculate a velocity for the object based on the determined impact energy of an impact event. In other applications, the velocity of the impact object may be known and the mass of the object could be calculated.

The impact analysis application 160 may include session records 162. The session records 162 may be recorded information for a history of impact events. In other words, the analysis performed on an impact event may be recorded in the session records 162. In this manner, the impact analysis application 160 can provide a historical analysis of impact events occurring on the impact apparatus 110. In some implementations, the session records 162 may be associated with a particular user. For example, session records 162 can be associated with different members of an athletic team (e.g., baseball team, football team, hockey team, etc.) and, thus, be associated with a player/athlete (i.e., user) identifier. In such implementations, session records (also referred to as history data) may be associated with particular users. In some implementations, the session records 162 may be associated with an object identifier. Thus, for example, different histories may be associated with different objects projected at (e.g., thrown at, kicked toward, hit toward, etc.) the impact apparatus 110. These object-histories can also be associated with a particular user identifier. In some implementations, a session is associated with a session identifier. Such an implementation enables a user to have multiple sessions. The multiple sessions can occur on different days, at different hours, etc. In some implementations, a user can choose to continue a session from a prior day or start a new session. In some implementations, the session records 162 are deleted when a new session is started. The content of and/or lifecycle of the session records 162 is implementation dependent, e.g., determined by the activity intended for the impact apparatus 110 and/or the user interfaces generated by the impact analysis application 160.

The impact analysis application 160 may include profile data 168. The profile data 168 may include information for scoring impact events representing an exercise on the impact apparatus 110. In some implementations, the profile data 168 includes weights to be assigned to the impact zones 105. As one non-exhaustive example, the impact apparatus 110 may be a tackling sled, tackling dummy, or padding worn by an opponent. The profile data 168 may reflect preferred impact zones 105 for a particular kind of exercise, e.g., a particular tackle, take-down, strike or strike combination. In other words, the profile data 168 may reflect weights that indicate whether the player is performing the exercise (tackling/striking/impacting) with proper technique, (e.g., hand/shoulder/limb placement is correct, and the helmet/head doesn't contact the dummy). The impact zones on the impact apparatus 110 expected to be impacted with proper technique are referred to as preferred impact zones or target impact zones. These target impact zones can be identified in a profile for the exercise in profile data 168.

In some implementations, the profile for an exercise may identify target impact zones and non-target impact zones. In some implementations, the profile for an exercise may identify the target impact zones by identifier. In some implementations, the profile for an exercise may identify target impact zones with a flag. In some implementations, the profile for an exercise may identify the target impact zones by assigning these zones a weight of one (1) and the non-target impact zones by assigning these zones a weight of zero (0). In some implementations, the profile for an exercise may identify the target impact zones and the non-target impact zones by weights assigned to the impact zones. For example, target impact zones may be assigned respective weight values that are positive and non-target impact zones may be assigned respective weight values that are negative. In some implementations, the profile for an exercise may identify the target impact zones and non-target impact zones by a flag or identifier and assign each of the impact zones a respective weight. In some implementations, the profile may define target and non-target impact zones and have two or more expertise levels, where the expertise levels determine the respective weights. In some implementations, a first profile may define/identify target zones and weights for a beginning expertise level of an activity and a second profile may define/identify target zones and weights for an expert or pro expertise level of the same activity. The target zones of the first profile may differ than the target zones of the second profile. The weights of the first profile may differ from the weights of the second profile.

In some implementations, a profile in the profile data 168 may be for an object of known mass and may include information for scoring impacts based on the determined magnitude of an impact event and/or an impact type determined for the impact event. For example, where the impact apparatus is padding worn by a user and various discharging devices fire, shoot, etc., objects (e.g., guns firing non-lethal projectiles), the objects may all be of similar mass, but the discharging devices may shoot the projectiles at different velocities at the impact apparatus. The different velocities register as different impact energies (as described herein) and can be used to classify or determine which discharge device was used. Scoring can be based on the types of devices used, in addition to which impact zone is hit. For example, the profile may indicate a different weight for different discharging devices. The profile may indicate different weights for the different discharging devices for the different impact zones. The profile may indicate different weights for glancing impacts as opposed to direct impacts, as disclosed herein.

The determination of the type of discharge device attributable to an impact and/or the determination of whether an impact event is a glancing impact or a direct impact may be performed by impact logic 124, impact analysis logic 164, and/or impact analysis application 160. For example, a larger discharging device may fire the projectiles with higher velocity but be physically large, so better for long-range use, where a smaller, lighter discharging device may operate more easily in closer range, but fire the object with a lower velocity. Thus, a first discharging device may be associated with a first velocity and a second discharging device may be associated with a second velocity. In some implementations, a classifier (e.g., a machine-learned model) can be used to analyze the voltage data to determine whether an impact event is attributable to a first discharging device or a second discharging device based on the magnitude (impact energy) of the impact event. Further, a projectile loses velocity the further it is away from the projectile device, if one projectile device is used and the mass is constant the system could determine the distance the projectile devices is from the target by the velocity of impact.

In some implementations, the system may determine whether an impact event is a direct impact or a glancing impact. For example, in the impact apparatus just described, although the projectiles are of a known mass and the discharging device projects the mass with a predictable velocity, this velocity may be associated with a direct hit. In other words, the voltage data generated during an impact event that is a direct impact may be used to correctly calculate the velocity (as disclosed herein). But an impact event that is not a direct impact event, e.g., represents a glancing blow or glancing impact, may have voltage data that differs in characteristic voltage trace shape. To account for this, the system may use voltage profile information to first determine an impact type for the impact event, e.g., whether an impact event is a direct impact or a glancing impact. For example, if the voltage data fits a first profile (e.g., a short spike over the impact period) it may be classified as a direct hit but if the voltage data fits a second profile (e.g., an impact event where the voltage data resembles more of a wide hill over the impact period), the impact event may be classified as a glancing impact. The magnitude of the glancing impact from a first discharging device may still differ from the magnitude of a glancing impact of a second discharging device due to the difference in velocity. Thus, the system 100 may classify a type of impact before determining which type of discharging device to attribute to the impact event. A classifier can also be used to analyze the voltage data and determine the type of impact. In some implementations, the system may use a combined classifier, e.g., a classifier that takes the voltage data for an impact event as input and provides a predicted discharge device as output.

In some implementations, the impact analysis application 160 may include a user interface that enables a user of the impact analysis application 160 to define a new profile, which is added to the profile data 168. In some implementations, the activity represented by a profile may be a series of impact events, e.g., a boxing combination. In such implementations, a profile may include a series of target/non-target impact zones. Thus, an impact zone may be identified as a target impact zone for a first impact event in the series but as a non-target impact zone in a second impact event in the series. The impact analysis application 160 can use the profile data 168 to score an impact (or series of impacts), as discussed herein.

In addition to stored profiles for a particular exercise, the profile data 168 may also temporarily store a target impact zone selected by a user of the computing device 150. For example, a user may select one or more of the impact zones 105 as a preferred target zone for a next impact event. In some implementations, the user may select a secondary impact zone as a target zone. In such implementations, the secondary impact zone may have a lower weight than the primary target zone. In some implementations, the target zone (and, if selected, the secondary zone) selected by the user may be communicated to the impact apparatus 110, e.g., via target zone data 140. The impact apparatus 110 (e.g., microcontroller 120) may be configured to receive the target zone data 140, determine which impact zone(s) 105 are identified in the target zone data 140 and to change an appearance of those zones. For example, the impact apparatus 110 may be configured to use a feedback device 107 associated with a target impact zone 105 to change the appearance of the impact zone 105, as described above. The change in appearance may reflect the primary and secondary target zones. For example, the primary target zone may have a first color and the secondary target zone may have a second color, where the person using the system 100 understands that the first color represents a primary target. This setup may challenge the user to decide which of the two target zones to impact (strike/aim at). This change in appearance may last until an impact event is detected by the microcontroller 120. This change in appearance may last for a predetermined period if an impact event is not detected before the period ends. In some implementations, in response to the impact event, the impact apparatus 110 may be configured to change the appearance of an impact zone 105 (or zones) that generated a voltage in response to the impact event. In some implementations, the impact zone 105 that generated the highest detected voltage may receive a change in appearance (e.g., by activating the feedback device 107 associated with this impact zone). In some implementations, two or more of the impact zones 105 that generate detected voltages may receive a change in appearance (e.g., by activating the feedback devices 107 associated with these impact zones). This change in appearance (responsive to the impact event) may be temporary, e.g., lasting for a predetermined period of time after the impact event. In some implementations, two or more of the impact zones 105 that generate detected voltages my receive a change in appearance that indicates the magnitude of the impact event.

The components (e.g., modules, processors) of the computing device 150 can be configured to operate based on one or more platforms (e.g., one or more similar or different platforms) that can include one or more types of hardware, software, firmware, operating systems, runtime libraries, and/or so forth. In some implementations, the components of the computing device 150 can be configured to operate within a cluster of devices (e.g., a server farm). In such an implementation, the functionality and processing of the components of the computing device 150 can be distributed to several devices of the cluster of devices.

The components of the computing device 150 (e.g., the impact analysis application 160 of the computing device 150) can be, or can include, any type of hardware and/or software configured to analyze voltage data. For example, in some implementations, one or more portions of the impact analysis application 160 in FIG. 1 can be, or can include, a hardware-based module (e.g., a digital signal processor (DSP), a field programmable gate array (FPGA), a memory), a firmware module, and/or a software-based module (e.g., a module of computer code, a set of computer-readable instructions that can be executed at a computer). For example, in some implementations, one or more portions of the components of the computing device 150 can be, or can include, a software module configured for execution by at least one processor (not shown). In some implementations, the functionality of the components can be included in different modules and/or different components than those shown in FIG. 1.

In some embodiments, one or more of the components of the computing device 150 can be, or can include, processors configured to process instructions stored in a memory. For example, the impact analysis application 160 (and/or portions thereof) can be, or can include, a combination of a processor and a memory configured to execute instructions related to a process to implement one or more functions.

Although not shown, in some implementations, the components of the computing device 150, such as the impact analysis application 160 of the computing device 150, can be configured to operate within, for example, a data center, a cloud computing environment, a computer system, one or more server/host devices, and/or so forth, although such implementations may delay feedback response time. In some implementations, the components of the computing device 150 can be configured to operate within a network. Thus, the components of the computing device 150 or impact apparatus 110 can be configured to function within various types of network environments that can include one or more devices and/or one or more server devices. For example, the network can be, or can include, a local area network (LAN), a wide area network (WAN), and/or so forth. The network can be, or can include, a wireless network and/or wireless network implemented using, for example, gateway devices, bridges, switches, and/or so forth. The network can include one or more segments and/or can have portions based on various protocols such as Internet Protocol (IP) and/or a proprietary protocol. The network can include at least a portion of the Internet.

In some implementations, the memory 122 and/or the memory 458 can be any type of memory such as a random-access memory, a disk drive memory, flash memory, and/or so forth. In some implementations, the memory 122 and/or the memory 458 can be implemented as more than one memory component (e.g., more than one RAM component or disk drive memory) associated with the components of the impact apparatus 110 or the computing device 150. In some embodiments, the calibration data 166, the custom profile data 168, or the session records 162 (or a portion thereof) can be a remote database, a local database, a distributed database, a relational database, a hierarchical database, and/or so forth. As shown in FIG. 1, at least some portions of the calibration data 166 and/or transmitted voltage data 130 can be stored in memory (e.g., local memory, remote memory) of the computing device 150. In some embodiments, the memory can be, or can include, a memory shared by multiple devices such as computing device 150.

FIG. 2 is a schematic diagram of an example impact zone 205 of an impact apparatus, according to an implementation. The impact zone 205 is an example of any impact zone 105 of FIG. 1. The impact zone 205 comprises a foam sheet 202, conductive adhesive 204, electrode 206, and wire 203. The wire 203 conducts voltage generated by the foam sheet 202 in response to an impact event to a microcontroller 220. The microcontroller 220 is an example of microcontroller 120 of FIG. 1. In some implementations, the impact zone 205 may comprise a separate foam sheet 202 that generates a voltage response proportional to impact energy when impacted (deformed). The foam sheet 202 includes a polymeric foam. The foam may be any polymeric foam, such as an elastomeric polymer foam, a silicone-based foam, a polyurethane foam, a thermoset foam, or other foam-like material. The foam may retain its shape after deformation, e.g., the foam may be capable of experiencing substantial deformations while substantially retaining its shape. In other words, the foam has elasticity, porosity, and high failure strain, typically from 50% to 100% strain. In some implementations, the adhesive, electrode, and wire may be combined by applying a conductive paint, a conductive ink, or other conductive coating that transmits the voltage data to the microcontroller 220.

In some implementations, the foam sheet 202 may include conductive fillers dispersed in the foam. Thus, in some implementations, the foam sheet 202 may be a composite material that includes conductive elements dispersed throughout the foam. For example, microscopic conductive elements, such as conductive fibers and/or nanoparticles may be included in the foam before curing to produce foam sheet 202. These conductive fillers can be a small proportion of the foam sheet 202, e.g., constituting less than 25% by weight. In some implementations, the conductive fillers may be a very small proportion of the foam sheet 202, e.g., less than 1% by weight, including 0.1% by weight. Some implementations do not have any conductive fillers added to the foam sheet 202.

In some implementations, the impact zone 205 may be defined by the location of wires 203 on a single foam sheet 202. Via the wire 203, conductive adhesive 204, and electrode 206, or the conductive coating, the impact zone may be operatively coupled to a voltage detector, e.g., in microcontroller 220. The wire 203, adhesive 204, and electrode 206 or conductive coating may be collectively referred to as conductive elements. As used herein, a conductive element includes conductive films, metals, printed circuits, or wires adhered to the foam sheet 202. Thus, the conductive elements conduct the voltage generated at impact by the foam sheet 202 to the microcontroller 220. The wire 203, the electrode 206 and the conductive adhesive 204 can be made of any conductive material, i.e., any material that conducts electricity. The conductive material can include metal, carbon, or other conductive material. The conductive elements are thus in contact with the foam sheet 202 and configured to convey voltage generated to the microcontroller 220. In some implementations, the electrode 206 can be a metallic coated film, sheet, or fabric that can be shaped in order to increase or decrease a material property of the foam sheet 202 (i.e., increase stiffness in one direction).

The conductive elements conduct the voltage generated at impact by the foam sheet 202 to a voltage detector, e.g., in microcontroller 220. Within the microcontroller 220, the voltage detector is operably coupled with a memory so that the voltage data (electric potential information) generated in response to an impact event on or near a sensor is recorded in the memory. The voltage data may be recorded for an impact event. An impact event is a period of time in which detectable voltage is measured. For example, when the foam sheet 202 is impacted, the foam sheet 202 creates (generates) a small voltage. This voltage may be sent via the conductive elements to the microcontroller 220. The microcontroller 220 may include components such as an inverting operational amplifier and analog to digital converter. The generated voltage may be sent through the inverting operational amplifier and then read by the analog to digital converter. The analog to digital converter can be configured to sample the voltage data at a sampling rate. In some implementations, the sampling rate can be 1,000 samples per second. The sampling rate can be adjusted to be faster or slower depending on desired precision and data transfer limitations. In some implementations, the microcontroller 220 can be configured to compress the samples. For example, a moving average or other compression methods can be used to down sample the 1,000 Hz measurements to a lower frequency, such as 200 Hz, for faster broadcasting to BLUETOOTH connected devices. In some implementations, the microcontroller 220 can also be equipped with flash memory allowing for raw data to be recorded for post analysis or post activity syncing. This voltage data (e.g., sampled at 1,000 Hz or compressed to 200 Hz, etc.) can be transmitted to a computing device with more processing power for further analysis. This keeps the form factor of the impact apparatus small. For each impact apparatus, an application on the receiving computing device will allow the user to view the results of each impact with immediate feedback. In other words, an entire system is configured for real-time feedback, e.g., with less than 1 second between detection of an impact event and feedback provided to the user, e.g., in the form of the location impacted and/or a magnitude of the impact. Impact zone 205 is one non-limiting example of an impact zone 105, but implementations can include an impact zone comprising any sensor that, in response to an impact, generates a voltage that is proportional to impact energy.

The impact zone 205 can include feedback device 207. The feedback device 207 can be any device giving a visual cue to a user conveying information about the impact zone 205. The information may indicate the impact zone 205 is a target impact zone. The information may indicate the impact zone 205 is a hit impact zone. The information may indicate the impact zone 205 is a secondary target impact zone. The information may indicate the impact zone 205 is a missed impact zone (e.g., a target impact zone that was not the hit impact zone). The feedback device 207 can include LED light strips, an LED backlight, or any other device capable of/configured to change an appearance to the impact zone 205.

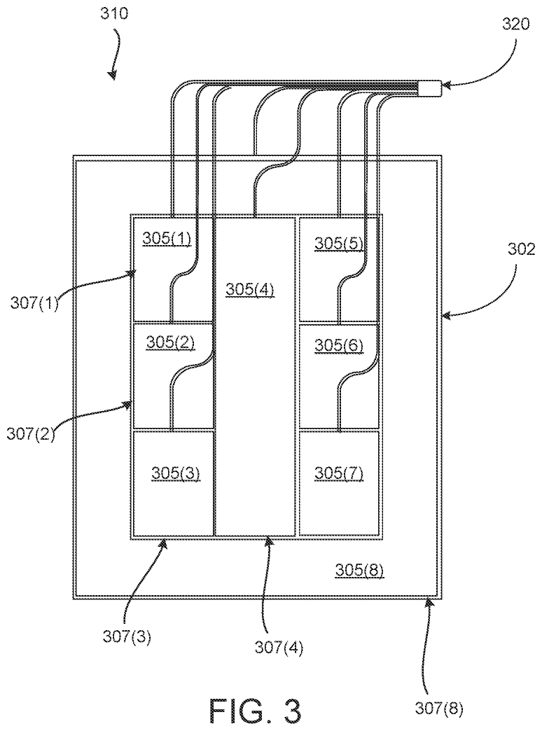

FIG. 3 is a schematic diagram of an example impact apparatus 310 with a plurality of impact zones 305, according to an implementation. The impact apparatus 310 is one example of an impact apparatus 110 of FIG. 1. The example impact apparatus 310 of FIG. 3 is described as a pitching target, but this is for purposes of explanation only. The impact apparatus 310 could be adapted to represent other kinds of sports targets, such as a hockey goal, soccer goal, golf driving backstop, tennis backstop, a mat covering one side of a volleyball court, etc.

In the example of FIG. 3, the impact apparatus 310 comprises a single foam sheet 302, In an example implementation, the foam sheet 302 is placed over several different conductive electrodes that are adhered to a firm backing. The electrodes are arranged so each electrode will measure an impact in a certain impact zone 305. A visual of the outline of the electrodes are shown in FIG. 3. Each separate electrode, which is connected to the foam sheet 302 (e.g., via a conductive adhesive), has a single conductive trace that is connected to the microcontroller 320 to allow for data acquisition (i.e., voltage generated in response to an impact). This conductive trace can create a noisy signal if popper considerations for static interference are not taken. These noisy signals create unreliable data and lead to less accurate measurements. Properly designed wires can reduce the noise and allow for a more accurate sensor measurement. Shielded wires (insulated wires) have been shown to decrease the noise significantly.

In the example of FIG. 3, the impact zones 305 are separated into a strike zone and an area outside of the strike zone (the ball zone). The ball zone is illustrated in FIG. 3 as impact zone 305(8). In some implementations, the strike zone can be divided into multiple impact zones. The impact zones can be of equal area. For example, the strike zone can be partitioned into three columns (or three rows). These portions can be further divided, e.g., each column could include three impact zones. Depending on the configuration, the impact zones 305 can be of equal area or one or more can be of different areas. For example, the impact apparatus 310 includes seven impact zones 305, e.g., impact zone 305(1) to impact zone 305(7). In the example of FIG. 3, the left and right columns each have three electrodes (the left column corresponding to impact zones 305(1) to 305(3) and the right column corresponding to impact zones 305(5) to 305(7)), and the middle column (corresponding to impact zone 305(4)) has a single electrode that stretches the entire height of the strike zone. The impact zones 305 are thus defined by an area sensed by conductive elements (electrodes/wires/conductive film). In the example of FIG. 3, the ball zone e.g., impact zone 305(8), surrounds the strike zone (e.g., columns defined by impact zones 305(1) to 305(7)). Other implementations may have more or fewer zones, but increasing the zones increases the cost of the target, while decreasing the zones decreases spatial resolution precision (i.e., reduces the precision with which the system can determine the location of the impact; if the spatial resolution is low enough, in some implementations, this could also affect the ability of the system to determine the impact energy). Accordingly, the number of zones is a balance between precision and cost. The configuration of the impact zones of the impact apparatus can be adapted to an activity (sport, exercise, etc.) simulated or performed using the impact apparatus. The impact apparatus 310 can determine if an impact has occurred by setting a flag when a voltage threshold is reached on any electrode for an impact zone 305. The setting of this flag may define the start of an impact event. In some implementations, the impact event lasts for a predetermined amount of time, referred to as an impact period.

In some implementations, an impact zone 305 may have a corresponding feedback device 307. In some implementations, not every impact zone 305 may have a corresponding feedback device 307. For example, impact zone 305(8) (the ball zone) may lack a corresponding feedback device 307, while impact zones 305(1) to 305(8) (the strike zone) may each have a corresponding feedback device 307, e.g., feedback devices 307(1) to 307(7). Some implementations include no feedback devices.

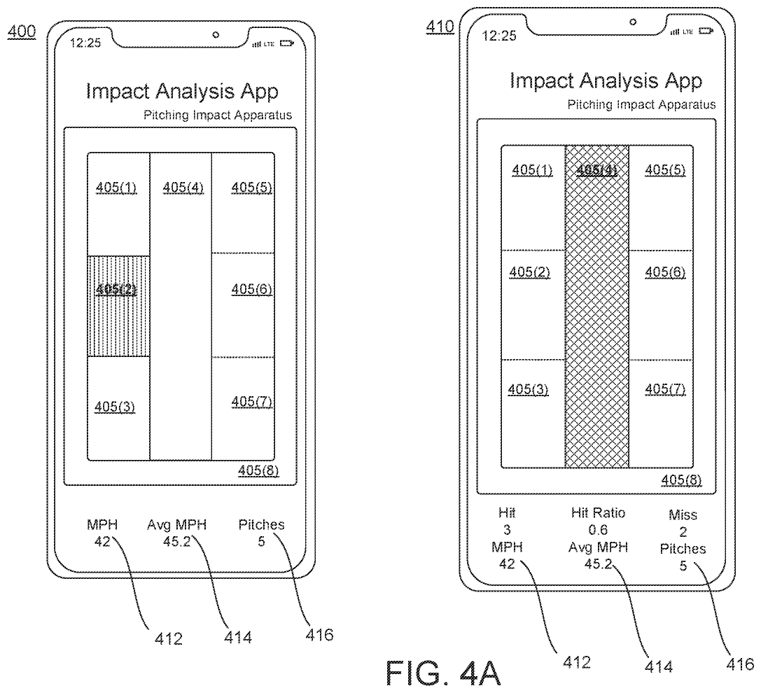

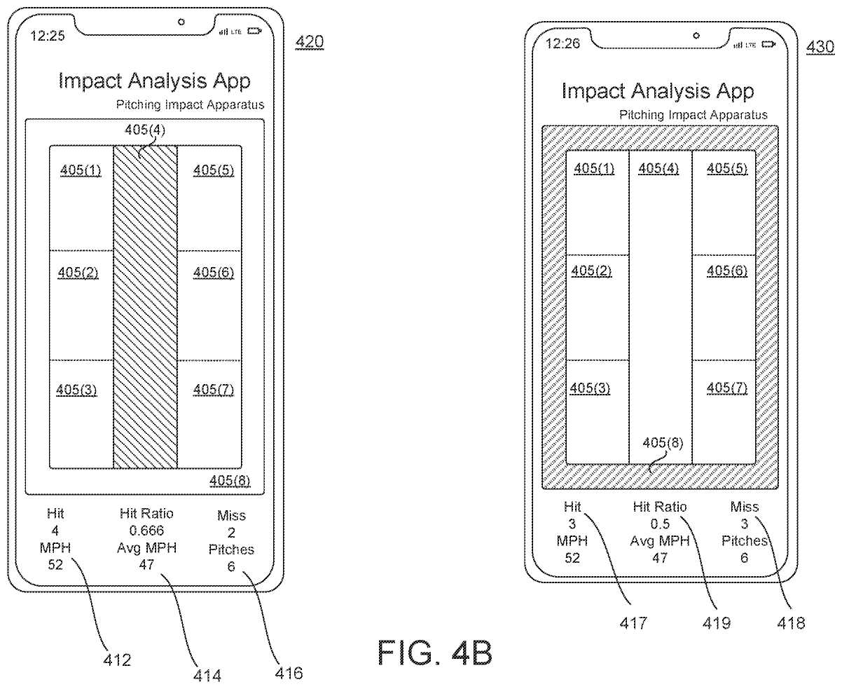

FIGS. 4A and 4B illustrate example user interfaces configured to interact with and provide feedback for the impact apparatus 310 of FIG. 3, according to an implementation. The user interfaces of FIGS. 4A and 4B may be generated by an impact analysis application (e.g., impact analysis application 160) of a computing device (e.g., computing device 150) in communication with impact apparatus 310. The user interfaces 400, 410, 420, 430 include a plurality of impact zones, e.g., virtual impact zones that correspond to the impact zones of the impact apparatus, e.g., physical impact zones. In the example interfaces of FIGS. 4A and 4B, the virtual impact zones 405(1) to 405(8) correspond to the impact zones 305(1) to 305(8) of FIG. 3.

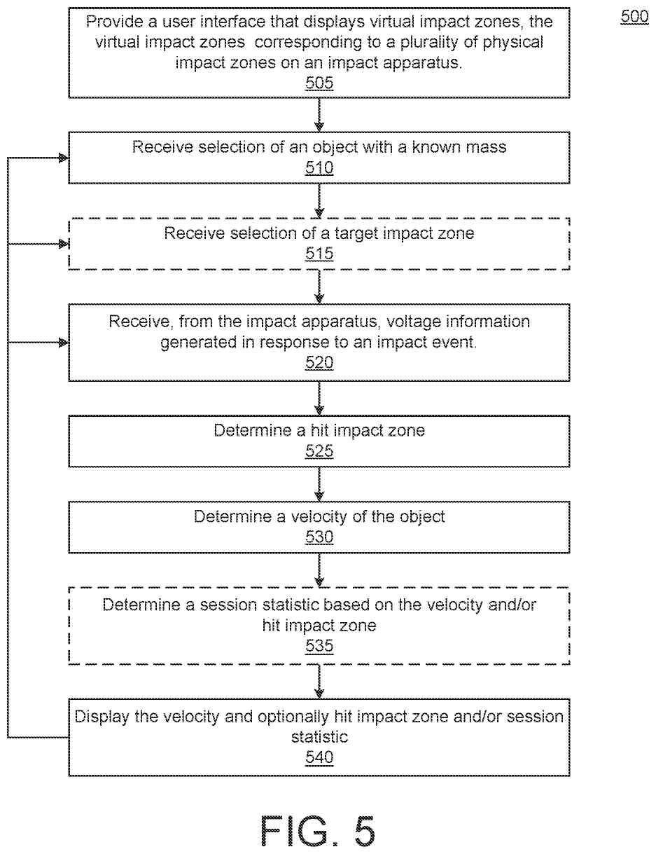

Once the system registers an impact (e.g., a threshold voltage detected at one of the impact zones on the impact apparatus), the voltage information may be analyzed as described in more detail with respect to FIG. 5. The impact analysis application may update the user interface as a result of the analysis, e.g., as illustrated in user interface 400. For example, the user interface may be updated to display the hit impact zone, e.g., impact zone 405(2). The hit impact zone is the zone registering the highest voltage. In the example of FIG. 4A, the hit impact zone is impact zone 405(2), which may have an appearance that indicates this is the hit impact zone for the impact event. Any difference in appearance may be used to indicate a hit impact zone. In some implementations, the impact apparatus itself may also update the appearance of the hit impact zone (e.g., the physical impact zone), as described elsewhere. The user interface 400 may also be updated to display the velocity 412 of an object causing the impact where the mass of the object is known. The impact analysis application may include a user interface (e.g., such as a settings option, not shown) for selecting known objects, e.g., a regulation baseball, a regulation softball, a regulation hockey puck, a regulation volleyball, etc.

In addition to displaying which impact zone was hit and with what velocity 412, implementations may enable a user (e.g., a coach or catcher) to “call” a zone, or in other words to signal to a pitcher which of the impact zones the pitcher should attempt to hit. In some implementations, the called zone may be selected by the coach or catcher via a user interface (e.g., selection of an impact zone displayed in the user interface). Thus, in some implementations, the impact zones 405 may be selectable. In other words, in some implementations, a user may select an impact zone of the impact zones 405 as a called or target impact zone. User interface 410 illustrates an example user interface with a selected impact zone, e.g., target impact zone 405(4). The user may select an impact zone as a target impact zone by touch (e.g., touching the impact zone on the user interface 400) or selecting with a mouse or other input device. In some implementations, the system (e.g., impact analysis application and/or impact analysis logic) may be configured to receive the target zone via voice command. In some implementations, the user interface may be configured to change an appearance of the selected impact zone, e.g., illustrated by the cross-hatch fill of impact zone 405(4) of user interface 410. The target change in appearance is feedback for which impact zone is the target zone. In an example user interface, a target zone may be changed to (be represented by) a first color, such as blue. Confirmation of a target zone can also be accompanied by feedback from other feedback devices, such as an audio signal played. In some implementations, the target impact zone selected by the user is communicated to the impact apparatus, which may be configured to change an appearance of the target impact zone, e.g., to temporarily change an appearance of the target impact zone on the impact apparatus. In some implementations, the system may be configured to flash the first color in the called zone at the impact apparatus. For example, instead of a coach verbally communicating the called zone, the coach may select the called zone via the user interface 410 and the impact analysis application may communicate the target impact zone to the impact apparatus, which may temporarily change an appearance of the target zone. The identification of a target impact zone is optional, and the user interface may be used to provide the output of an impact event without receiving a target impact zone.

If the next impact event is in the target impact zone, the system may record that the correct zone was hit, i.e., that the hit impact zone matches the target impact zone. For example, a second color may be used to indicate the hit impact zone matches the target impact zone, while a third color may be used to indicate the hit impact zone differs from the target impact zone. In some implementations, the user interface may display or flash the second color if the target zone is hit. For example, when the hit zone matches the target zone, the user interface may display or flash green. In some implementations, the hit zone is displayed in green in the user interface. User interface 420 illustrates a target impact zone (405(4)) that was both the hit impact zone and the target impact zone (e.g., the target zone 405(4) of user interface 410).

If the next impact is not in the target zone (i.e., the target impact zone does not match the hit impact zone), the system may display or flash a third color, e.g., such as red. In some implementations, this third color may be displayed in either the target impact zone or the hit impact zone. User interface 430 illustrates an example user interface where a hit impact zone 405(8) is not the target impact zone (e.g., impact zone 405(4) and the hit impact zone 405(8) has its appearance changed with the third color. An implementation that changes the appearance of the missed target impact zone would look similar to user interface 420, but with the third color. In some implementations, if the target impact zone differs from the hit impact zone, the target impact zone may have an appearance that differs from the hit impact zone. For example, user interface 430 could be rendered with impact zone 405(4) shaded in the first color (e.g., as in user interface 410), or with a fourth color, such as gray, etc. In some implementations, the impact apparatus itself may change an appearance of the hit impact zone or target impact zone (e.g., via feedback devices 107) to communicate the hit impact zone, the hit impact zone matching the target impact zone (e.g., flashing green for the hit impact zone), and/or the hit impact zone being different from the target impact zone (e.g., flashing red for the hit impact zone or for the target impact zone). Instead of colors, the system may provide audible indications of whether a called zone was hit or not. User interfaces 420 and 430 also illustrate updating the velocity 412 of the most recent impact event.

Some implementations may keep and display session statistics. For example, analysis for a history of impact events may be kept and may be used to provide session statistics. These session statistics can include the number of impact events 416 in the session. The session statistics can include the average velocity 414 over the number of impact events 416. User interfaces 420 and 430 also illustrate updating the average velocity 414 and impact events 416 using the most recent impact event.

For sessions that include a called or target impact zone, the session statistics can include hits 417 and/or misses 418. The hits 417 represent the total number (count) of times the hit impact zone matched the target impact zone during the session. The misses 418 represents the total number (count) of times the hit impact zone does not match the target zone. In some implementations, the session statistics may include a hit ratio 419. The hit ratio 419 can be calculated from the total number of impact events 416 and either the hits 417 or the misses 418. In some implementations, the hits 417 or misses 418 may be calculated using the total number of impact events 416. In some implementations, the impact events 416 may be calculated from the hits 417 and the misses 418. In other words, the system may store only two of the hits 417, the misses 418 and the impact events 416, as one of these values can be calculated from the other two and the hit ratio 419 can be calculated from any two of the three. A user may start a new session (and thus initialize stored session data) via a user command, selection of another object with a different known mass, etc.

FIG. 5 is a flowchart that illustrates an example process 500 for determining velocity of an object striking an impact zone of an impact apparatus, according to an implementation. The system performing the process may be the system 100 of FIG. 1. For example, the steps of the process 500 can be performed by any of the impact analysis logic 124, impact analysis logic 164, and/or impact analysis application 160. The process 500 of FIG. 5 can be performed to provide a velocity of an object impacting the impact apparatus.

While the impact apparatus of FIG. 3 and the user interfaces of FIGS. 4A and 4B are described with respect to a pitching target, the user interfaces can be adapted to other activities. For example, in one implementation the impact apparatus may be a shooting target. Projectiles of known mass may be selected via the user interface. The impact apparatus may be comprised of several impact zones, e.g., electrodes' interfaces placed between two pieces of piezeo-electric foam. This arrangement may then be placed between two sheets of metal suitable for withstanding penetration from large caliber rounds. The conductive interfaces between the foam each have conductive traces connected to the microcontroller for data acquisition. When this impact apparatus is shot, the foam sheet will produce a voltage that will be sent to the microcontroller allowing the user to immediately (e.g., in real-time) know hit location when utilizing a Bluetooth enabled (or other wireless) device and application. In addition, if the mass of the projectile is known, the velocity may also be provided via the user interface.

Other implementations include a target that measures velocity and location of a volleyball spike or serve, a softball pitching target that measures location and speed of pitch, a lacrosse target that fits the size of a goal and gives location and speed of shot, a hockey target that fits the size of a goal and gives location and speed of shot, a soccer target that will give location and speed of kick, a golf target that gives the location and speed of a shot, etc. Generally, implementations can include an impact apparatus having a piezo-electric foam base with electrodes in an optimal configuration adhered to the foam. This configuration allows for the accurate detection and measurement of an impact and, when the mass of the projectile is known, accurate velocity of the projectile upon impact.

The process 500 may begin by providing a user interface that displays virtual impact zones, the virtual impact zones corresponding to a plurality of physical impact zones on an impact apparatus (505). For implementations that calculate velocity, the system receives selection of an object with a known mass (510). In some implementations, this may be a fixed value (e.g., an application for a volleyball target, where it is assumed that the volleyball is a regulation volleyball of a known mass). In some implementations, the system may provide a setting or selection menu for selecting an object. For example, a pitching application may allow for selection of a baseball or a softball. In some implementations, the system may enable a user to provide a mass for the object.

In some implementations, the system may receive selection of a target impact zone (515) via the user interface. As used herein, target impact zone(s) are selected from among the virtual impact zones, but because each virtual impact zone directly corresponds to a physical impact zone, reference to a target impact zone includes the physical impact zone corresponding to the virtual impact zone selected as the target impact zone. Accordingly, transmitting a target impact zone to the impact apparatus is understood to mean that an identifier is transmitted to the impact apparatus, which is configured to translate the identifier to a physical impact zone (e.g., the electrodes corresponding to the impact zone, an area of the impact apparatus corresponding to the impact zone, etc.). Likewise, transmitting a sequence or series of target impact zones is understood to be transmission of identifiers for those zones, which the impact apparatus is configured to convert/map to the physical impact zones (strain sensors).

The system then receives, from the impact apparatus, voltage information generated in response to an impact event (520). In some implementations, the voltage information is generated (in whole or in part) by impact analysis logic. The impact analysis logic may be included in a microcontroller of the impact apparatus. The impact analysis logic may be included in a computing device communicatively coupled to the impact apparatus.

In some implementations, the location of the impact is determined by the location of the impact zone (e.g., the electrode for the impact zone) that registered the largest voltage response. This determination can be made at the impact apparatus (e.g., by impact analysis logic 124) and communicated to an impact analysis application or at the computing device (e.g., impact analysis logic 164). In either case, the hit impact zone is determined (525). The hit impact zone is the location of the impact event. Similarly, a velocity of the object may be determined from the voltage information (530).

The velocity may be determined after determining a magnitude of the impact. This magnitude directly correlates to the impact energy (e.g., using calibration data 166) because the impact zones produce a voltage that directly correlates to impact energy. When the piezoelectric foam of the impact zone is impacted, it generates a quantifiable voltage that changes magnitude with respect to time over an impact period. The impact period is short, e.g., less than a second. In some implementations, the impact period may be a 0.2 second period or a 0.15 second period. The length of the impact period can be determined by a number of factors, including the thickness and stiffness of the foam and/or the properties of the expected projectile. Generally, the impact period is determined during manufacturing of the impact apparatus by observing test impacts under expected conditions Generally, the impact period reflects the expected time span of the voltage response observed under expected use conditions.

To quantify the energy of impact, the system may be configured to measure the peak voltage at the impact and the integral of the voltage trace for several different points preceding and following the peak voltage. For example, the system may determine the peak voltage of the impact period (e.g., 0.15-second period), and align the peak voltage at the 0.05-second timestamp. With this alignment, the system may determine, the integral from 0s to 0.05s, the integral from 0.05s to 0.06s, the integral from 0.05s to 0.07s, the integral from 0.05s to 0.08s, the integral from 0.05s to 0.09s, the integral from 0.05s to 0.1s, and the integral from 0.05 to 0.15s. Based on the values of all these variables, the system can accurately predict the impact energy (magnitude) of an impact event. In some implementations, a regression model may be used to analyze the voltage data and provide the impact energy for the impact event. With the impact energy, which is a direct transformation of kinetic energy, the system can determine the velocity of the object by rearranging the following equation:

$${E}_{t} = \frac{1}{2} {mv}^{2} as v = \sqrt{2 \frac{{E}_{t}}{m}} ,$$

where m is the known mass of the object and Et is the impact energy. If an impact event registers on more than one impact zone (e.g., more than one impact zone has a voltage response that meets a threshold), the magnitude measured by each impact zone may be calculated and summed to determine the magnitude of the impact event. In this scenario, the impact zone with the highest magnitude is considered the hit impact zone. If only one impact is expected the system can sum the signal from two or more adjacent impact zones to determine a total impact that bridges multiple zones. In some implementations, this total impact is used to determine impact energy/velocity, etc. In some implementations, any impact zone (or all impact zones) with an attributable voltage may be considered a hit impact zone.

In some implementations, the system may be configured to determine session statistics based on the velocity and/or the determined hit impact zone (535). The session statistics can include the total number of impact events occurring during a session. The session statistics can include the average velocity over the session. The session statistics can include the mean velocity over the session, or other statistical operations (quartiles, etc.) applied to the session data. A session may be defined by the user. In some implementations, selection of a new object triggers a new session. In some implementations, session data can be associated with a time period (e.g., all impacts for a certain hour, for a certain day, etc. In some implementations, session data can further be associated with a user (e.g., a particular player). When a new session is started, the session data starts the total impact events at zero, so that all other statistics are zero. In other words, session statistics are initialized at the start of a new session.

The system may update the user interface to display the velocity and/or the hit impact zone (540). Updating the user interface with the hit impact zone may include providing an indication of whether the hit impact zone matches the target impact zone. In implementations, the system updates the user interface with updated session statistics. It is understood that steps 520 to 540 can be repeated several times. In implementations that include selection of a target impact zone, steps 515 to 540 can be repeated several times.

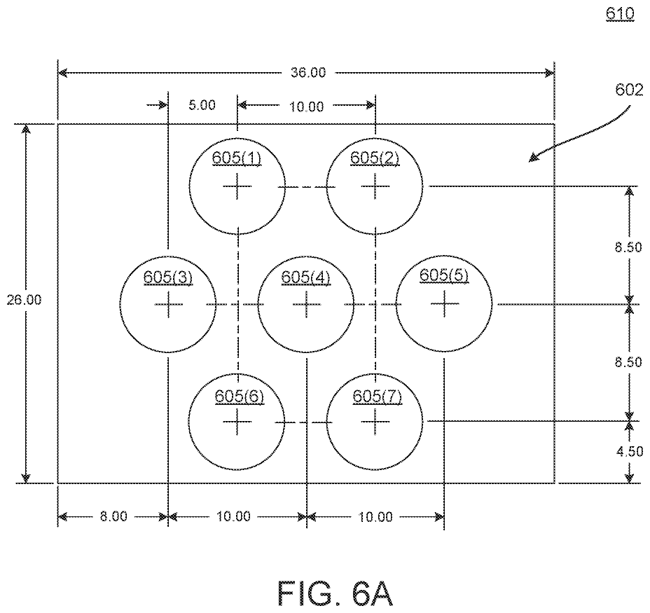

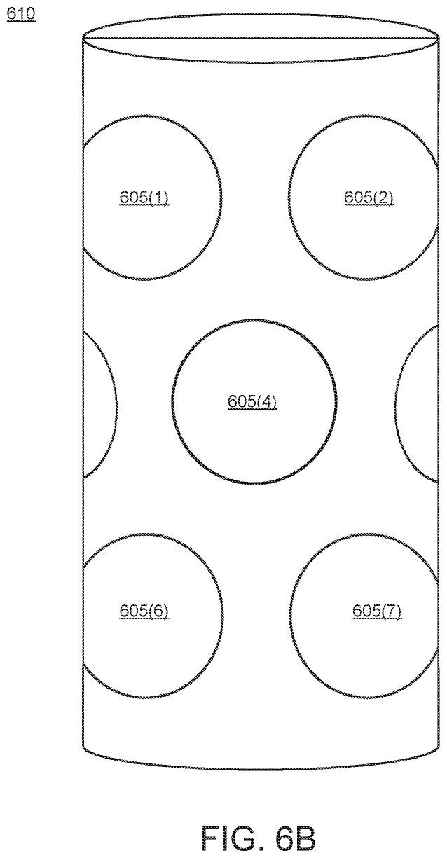

FIG. 6A is a schematic diagram of an example impact apparatus 610 with a plurality of impact zones, according to an implementation. The impact apparatus 610 is one example of an impact apparatus 110 of FIG. 1. The example impact apparatus 610 of FIG. 6A is described as a punching bag, but this is for purposes of explanation only. In the example of FIG. 6A, the impact apparatus 610 is a removable sleeve configured to fit around a punching bag. The impact apparatus 610 may include several impact zones 605. In the example of FIG. 6A the impact apparatus 610 includes seven impact zones, e.g., impact zone 605(1) to impact zone 605(7). However, implementations can include more or less, depending on the size (circumference and/or length) of the bag as well as other factors. The impact apparatus 610 as illustrated is configured for a bag with a circumference of approximately 36 inches. As illustrated, the impact apparatus 610 has a height of 26 inches. However, these measurements are example measurements and implementations are not limited to these specifications.

The impact apparatus 610 can include a piezo-electric foam sheet 602. The impact apparatus 610 can include a number (e.g., five, seven, nine, 15, etc., depending on the dimensions of the bag for example) of conductive film electrodes adhered to the foam using conductive adhesive, thus defining a plurality of impact zones 605. FIG. 6A illustrates example spacing of the impact zones 605, although implementations are not limited to this spacing. The spacing can be configured for a particular activity, e.g., so that the impact zones are reachable from a front of the bag. In some implementations, the spacing may provide sufficient distance between pads to prevent multiple pads from being impacted simultaneously on accident, where such separation is desirable. Each electrode may have a single conductive trace which connects to a microcontroller (not shown). As with other described implementations, the conductive trace can be an insulated wire, which mitigates receiving unreliable data. The foam sheet 602 and electrodes (defining the impact zones 605) are placed inside a punching bag sleeve with the insulating wires leading to a microcontroller outside of the sleeve. The microcontroller may be an example of microcontroller 120 of FIG. 1. The microcontroller may control signal processing and data flow. The microcontroller may be co figured to determine the location and magnitude of the impact. When one of the impact zones 605 is impacted, the microcontroller can be configured to determine the time of the peak voltage and calculate the impacted energy, as described above with respect to FIG. 5.

In some implementations, the punching bag sleeve may have adjustable straps for affixing around the circumference of the bag, as well as adjustable straps that attach on the top of the sleeve. This allows for the punching bag sleeve (impact apparatus 610) to be used with any existing punching bag by strapping the impact apparatus 610 around the punching bag and over the top of the bag. It also allows the impact apparatus 610 to be positioned along the length of the bag, as needed, e.g., for kicks as opposed to punches or to adjust to the height of the boxer.

FIG. 6B is an illustration of the impact apparatus 610 of FIG. 6A affixed to a punching bag, according to an implementation. The surface of the impact apparatus 610 (e.g., the sleeve into which the foam sheet 602 and electrodes are inserted, may include markings to identify the impact zones 605. In some implementations, the markings may include one or more feedback devices (not shown), e.g., causing the impact zone to change appearance as described herein. In some implementations, recessed LEDs my surround the impact zones to provide feedback or signal the user without affecting the function of the padding. In some implementations, the impact zones 605 may be identified using identifications, e.g., numbers, letters, or symbols, printed on the sleeve.

A connected application (e.g., impact analysis application 160) can track a user's progress over time determining if the impact force of their exercises is increasing, if their accuracy is increasing, if their response time is decreasing, etc. In some implementations, the connected application may enable a user (e.g., a coach) to select a target impact zone, as described herein. In such implementations, the impact apparatus 610 may be configured to provide a visual indication (e.g., light flash) for a particular impact zone (e.g., using a feedback device corresponding to the zone). In some implementations, the system may record how fast the target impact zone is hit. The response time may be determined by measuring the time elapsed between a start time and a stop time. The start time may be when the coach provides a start signal—e.g., via an audible command, a visual feedback is provided on the bag itself, and/or through a secondary sensor system. The stop time may be when the dummy is impacted by the participant. For example, the system may be configured to start a timer (record a start time) in response to the selection of a target zone or in conjunction with the activation of the feedback device for the target zone. The timer can be stopped (a stop time recorded) in response to receipt of an impact to the target impact zone. In such implementations, a stop time may not be recorded until an impact event with the target impact zone as the hit impact zone is determined. In some implementations, the stop time may be recorded (the timer stopped) when a next impact event is detected, regardless of the zone. In such an implementation no credit (e.g., a zero score) may be given for the impact event because the target impact zone was not the hit impact zone. In addition, the system may determine a magnitude of the impact and/or an impact energy of the impact.

In some implementations, the connected application may enable a user to select a primary target zone and a secondary target zone. In such an implementation, the user may be expected to choose between the two impact zones. In some implementations, where impact events are scored, the primary impact zone may be weighted more than the secondary impact zone. The impact zones can be communicated via feedback devices associated with the impact zones or an audible indication configured to inform the boxer of the identification of the selected target impact zone (or the primary impact zone and the target impact zone). In some implementations, the audible indication may be identification of the symbols printed on the sleeve. In implementations where response time is determined, the timer may be started in response to or with the audible indication. Recording the time of the audible indication may be considered starting a timer. In some implementations, the user of the connected application may start a timer (record a start time) and give the target zone/primary and secondary target zones.

In some implementations, the user of the connected application may select a profile, e.g., from profile data 168. The profile may represent a series of target impact zones. Thus, the profile may represent a series of impact zone identifiers. For example, the profile may represent a punch (or kick, or punch/kick) combination to be completed by the boxer. In some implementations, the system may be configured to change the target impact zone after each detected impact event, e.g., changing to the next target impact zone in the series. In some implementations, the system may be configured to wait until the target impact zone is a hit impact zone before moving on to the next target impact zone in the series. In some implementations, the timer may be configured to determine a response time for each target impact zone in the series, e.g., determining how long it took the boxer to impact the correct target impact zone.

In one implementation, the sensors could be placed in an array that could be used for reflex training. The system could indicate a location to impact and calculate a score based on the response time (e.g., between the indication of the location and the contact with the location) and the magnitude of the contact. This could be repeated for the desired duration or number of events from user input. Although discussed with respect to a punching bag, the impact apparatus 610 could be adapted for placement on any surface, including but not limited to a punching bag, a martial arts training dummy, the wall, floor, and/or ceiling to train hand and foot reflexes. In some implementations, the impact zones can be configured to flash different colors, some colors signifying locations of higher point value. As indicated earlier, these colors may be presented at the same time, so the user has to decide between two different impact zones before striking.

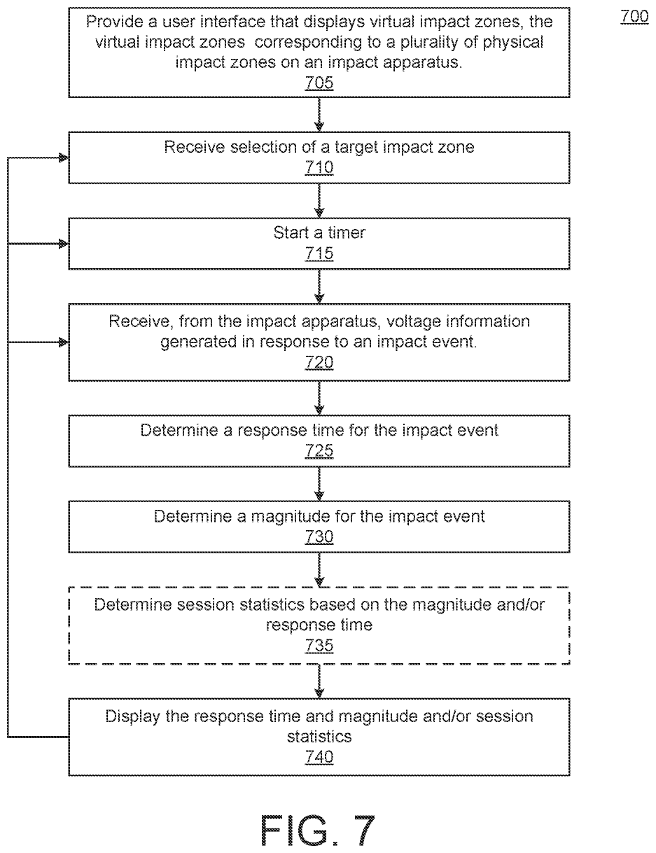

FIG. 7 is flowchart that illustrates an example process 700 for scoring impacts to an impact apparatus, according to an implementation. The system performing the process may be the system 100 of FIG. 1. For example, the steps of the process 700 can be performed by any of the impact analysis logic 124, impact analysis logic 164, and/or impact analysis application 160. The process 700 of FIG. 7 can be performed to provide a response time and impact magnitude for an object impacting the impact apparatus. As non-limiting examples, the process 700 could be used in an impact apparatus used in various activities, such as soccer, lacrosse, pitching, etc.

The process 700 may begin by providing a user interface that displays virtual impact zones, the virtual impact zones corresponding to a plurality of physical impact zones on an impact apparatus (705). The system may also receive selection of a target impact zone (710) via the user interface. The target impact zone may be selected by selection of a virtual impact zone displayed on the user interface. The target impact zone may be selected by selection of a profile, i.e., a series of target impact zones. The selection of the target impact zone may include receiving a primary target impact zone and a secondary target impact zone. In some implementations, a profile may include a primary target impact zone and a secondary target impact zone. Thus, in some implementations, one or more stages in the series may include two or more target zones. In some implementations, the profile may include weights to apply to the identified target impact zones. In some implementations, a primary target impact zone may have a higher weight than a secondary target impact zone. In a profile impact zones that are not target impact zones can have a zero weight or a negative weight.

The system may start a timer (715). The system may start the timer in response to selection of the target impact zone. The system may start the timer in response to a user command (including an audible command). The system may start a timer in response to (or in conjunction with) a change in the appearance of the target impact zone. The system may receive, from the impact apparatus, voltage information generated in response to an impact event (720). In some implementations, the voltage information is generated (in whole or in part) by impact analysis logic. The impact analysis logic may be included in a microcontroller of the impact apparatus. The impact analysis logic may be included in a computing device communicatively coupled to the impact apparatus. The voltage information can include the sampled voltage as described herein.

In some implementations, the voltage information is received in response to an impact event where the target impact zone is determined to be the location of the impact. This location may be determined as the location of the impact zone registering the largest voltage response. This determination can be made at the impact apparatus (e.g., by impact analysis logic 124) and communicated to an impact analysis application or at the computing device (e.g., impact analysis logic 164). In either case, a timer may be stopped in conjunction with the impact event on the target impact zone and a response time for the impact event is calculated (725). In some implementations, a magnitude for the impact event is determined (730). The magnitude directly correlates to the impact energy (e.g., using calibration data 166) because the impact zones produce a voltage that directly correlates to impact energy, as described with regard to FIG. 5. The magnitude may be represented as an average force, peak force, or impact energy.

In some implementations, the system may be configured to determine session statistics based on the magnitude, response time, etc. (735). The session statistics can include the total number of impact events occurring during a session. The session statistics can include the average response time over the session. The session statistics can include the mean response time over the session, or other statistical operations (quartiles, etc.) applied to the response times in the session data. The session statistics may include the average (and/or mean, and/or quartile) of the magnitude of the impact events during the session. As with FIG. 5, a session may be defined by the user. In some implementations, session data can be associated with (defined by) a time period (e.g., all impacts for a certain hour, for a certain day, etc. In some implementations, session data can further be associated with a user (e.g., a particular boxer). When a new session is started, the session data starts the total impact events at zero, so that all other statistics are zero. In other words, session statistics are initialized at the start of a new session.

The system may update the user interface to display the response time and/or the impact magnitude (740). In some implementations, the system may score the impact event, e.g., based on the response time (e.g., faster responses representing a higher score) and/or based on the magnitude (e.g., higher magnitudes representing a higher score). In some implementations, e.g., where target zones are assigned weights, the method may include determining which target impact zone is the hit impact zone and using the weight of the hit impact zone to determine the score. The system may update the user interface (and/or the session data) with this score. In some implementations, the user interface may graph the session data, e.g., showing how response times and/or magnitude is trending. It is understood that steps 710 to 740 can be repeated several times. In an implementation where the user selects a profile (e.g., a series of target impact zones), the system may repeat steps 710 to 735 automatically and update the user interface 740 once the sequence has completed (e.g., impact events are received and analyzed for the series of target impact zones). In such implementations, a response time can also be calculated for the overall sequence.

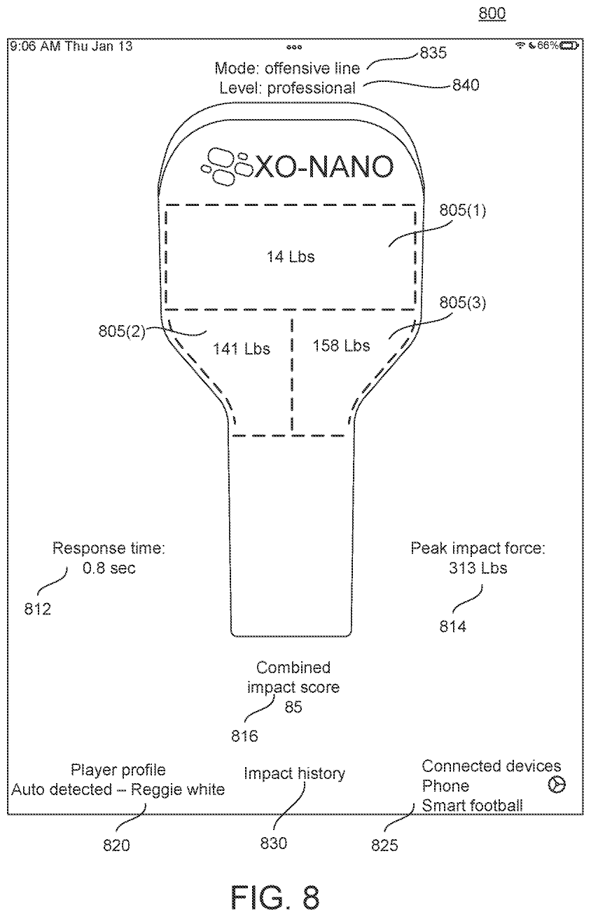

FIG. 8 illustrates an example user interface 800, according to an implementation. In the example of FIG. 8, the impact apparatus includes a training dummy. The training dummy of FIG. 8 includes three impact zones, e.g., impact zone 805(1), 805(2), and 805(3). Implementations are not limited to this number or configuration but are provided as an example. In some implementations, the impact apparatus may also include impact zones located in padding worn by a participant. For example, one or more impact zones included in the system may be placed in football training pads, rugby training pads, a helmet, etc. In addition, while the user interface 800 is described as for an impact apparatus representing a tackle sled, the impact apparatus can be configured as any sports training dummy, and can include impact zones located in boxing training pads, martial arts training pads, etc.

In an implementation where the impact apparatus represents a training dummy, the system may include multiple impact zones to indicate if the player is tackling/striking/impacting with proper technique, (e.g., hand placement is correct, and the helmet/head doesn't contact the dummy). In addition, the system may be configured to determine the magnitude of each impact event. In some implementations, the system may determine the response time of each impact event (e.g., tackle/strike). The response time may be determined by measuring the elapsed time between when the coach provides a start signal—e.g., via an audible command and/or through a secondary sensor system—and when the dummy is impacted by the participant. For example, a coach may provide an audible command such as “hike” or another selected command that the system recognizes as the start signal. In some implementations, the system can include an instrumented ball, e.g., a motion sensor connected to a football, or an instrumented stick connected to the ball. When the motion sensor measures motion associated with a predetermined/predefined motion, such as a “hike” motion, the sensor system may interpret the motion as a start signal and start a timer. In some implementations, the sensor system may communicate the start signal to the system, e.g., triggering the start of a timer. In some implementations, an instrumented training pad may be configured to communicate an end signal to the system. The instrumented training pad may be similar to an impact zone, but can be located on padding and not used in scoring the impact event. The instrumented training pad can be an impact zone, e.g., so that any detected impact event on an impact zone provides a stop signal to the system.

The system may then calculate the response time, e.g., as a difference between the start time (the start signal) and the stop time (the stop signal, or the time of contact with the instrumented training pad). The system may provide a response time to the participant via the user interface 800, e.g., response time 812. In some implementations, the system may also calculate a magnitude of the impact for each impact zone. For example, impact zone 805(1) is displayed as having a magnitude of 14 pounds, impact zone 805(2) a magnitude of 141 pounds, and impact zone 805(3) a magnitude of 158 pounds. In some implementations, the system may calculate, and the user interface may display an impact score 814. The impact score 814 may be a combination of the magnitudes determined for the impact zones.

In some implementations, the system may provide a combined score 816 of the response time and the impact score 814. The impact score can be based on impact magnitude and correct form. The impact score may be calculated based on “good” and “bad” impact zones. The “good” impact zones are target impact zones, which increase the combined score 816 when impacted. The “bad” impact zones are non-target impact zones, which reduce the combined score 806 when impacted. Each zone may have different scalers, e.g., positive or negative weights, to adjust/contribute to the score according to the level of “good/bad” form they represent. The good and bad impact zones and their scalers may be stored as a profile, e.g., in profile data 168. Thus, the combined score 806 may represent a weighted combination of scores from various impact zones. The system can have different modes/impact profiles to accommodate different types of impacts/tackles (e.g., training that is specific to different positions or roles on the team). The system (including the scalers) may be adjusted for different expertise levels of play 840, e.g., an expertise level ranging from youth to professional athletes.

A connected application, e.g., impact analysis application 160, can track a user's progress over time (e.g., during a session or a plurality of sessions), determining if their response time, form, and tackling/impact force is increasing. The session data can be accessed via impact history link 830. Some or all of the session data could be displayed (not shown in FIG. 8) as part of the user interface 800. This data could be tracked over time for each player by tracking which user is using the system through any means of proximity sensors (e.g., RFID, etc.). In the example of FIG. 8, the user interface 800 shows the automatic player detection 820 as well as a connected event detection in a football 825. The connected application is shown in Offensive line mode 835 where impacts to the top impact zone 805(1) are not desired, making impact zone 805(1) a non-target zone. The magnitude of the impact event attributed to top impact zone 805(1) may be provided in the user interface 800 with an appearance that indicates impact zone 805(1) is a non-target zone, e.g., using a first color (e.g., red font) to display the magnitude of 14 pounds or using a background of the virtual impact zone in the first color. The non-target impact zones may reduce the combined score 816. In the example of FIG. 8, the impact zone 805(1) may have a negative scaler, e.g., the recorded impact force of 14 pounds contributes to a lower combined impact score. In contrast, left impact zone 805(2) and right impact zone 805(3) are target impact zones. The appearance of these zones may reflect their status as target zones, e.g., with their recorded impact magnitudes of 141 pounds and 158 pounds respectively, displayed using a second color (e.g., green font) or the background of the target zones being represented in the second color.

Using the example of FIG. 8, the system may calculate the combined score in the offensive line mode as Combined Score=¹_{/Response time} (Left Force+Right Force−Top Force). The Left Force is the magnitude measured at the left impact zone 805(2), the Right Force is the magnitude attributable to the right impact zone 805(3), and the Top Force is the magnitude attributable to the top impact zone 805(1). A reciprocal of the response time can be used to increase the score for shorter response times. For another mode/impact profile, it may be desired that the right impact zone 805(3) or the left impact zone 805(4) have a higher recorded impact force than the other impact zone. This may be done using scalars (weights) applied to the magnitude. In such a scenario, the combined score may be expressed as Combined Score=¹_{/Response time} (w₁Left Force+w₂Right Force−w₃Top Force), where w₁ is a weight (scalar) given to the left impact zone 805(2), w₂ is a weight (scalar) given to the right impact zone 805(3), and w₃ is a weight given to the top impact zone 805(1). In some implementations, each weight may be customizable, e.g., by a user of the application to create or customize a mode/profile. As an example, an open field tackle profile may not have a response time associated with the score and the combined score could be calculated as: Combined Score=Σall forces.

In another mode/impact profile it may be advantageous to only have one impact zone positively affecting the combined score and the others reducing the combined score. In some implementations, the combined impact score may include logic that compares the recorded impact force between sensors, e.g., the combined score may be based on a difference between the Left Force and the Right Force depending on the mode/impact profile. For example, a profile/mode may indicate that the smaller the difference between the Left Force and the Right Force, the higher the contribution to the combined score. In some implementations, a time between a start signal and first impact may be factored into the combined impact score. In some implementations, response time may not be a factor.

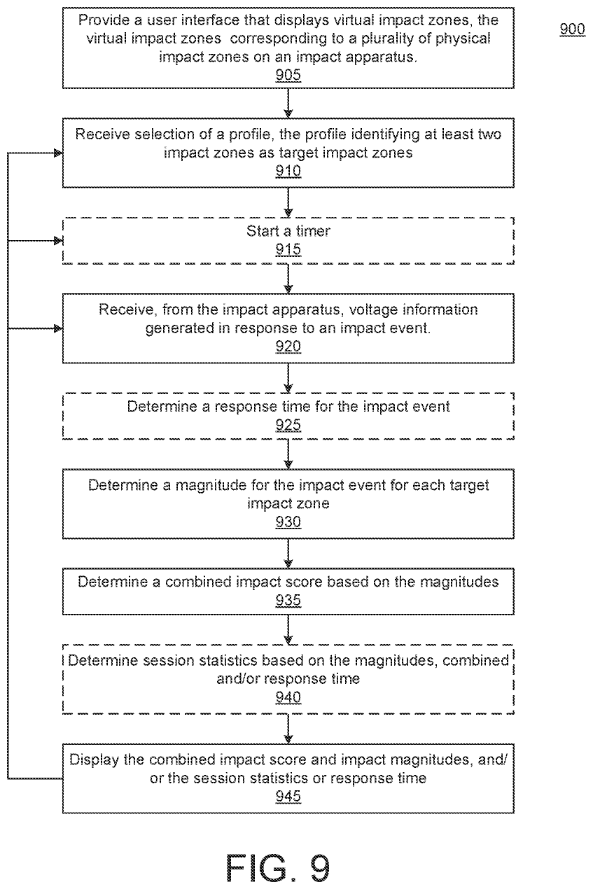

In some implementations, a player may be wearing a sensor, e.g., in a helmet, in a shoulder pad, etc., and a recorded impact force on the wearable sensors may contribute to the combined impact score (e.g., a helmet may have a negative potentially highly weighted scalar, while a shoulder pad may have a positive scalar; a left shoulder pad may have a negative scalar where the right shoulder pad has a positive scalar, or vice versa, depending on the mode/impact profile). In some implementations, the combined score 816 can be calculated as described with respect to FIG. 9.

FIG. 9 is a flowchart that illustrates an example process 900 for scoring impacts to an impact apparatus based on an impact profile, according to an implementation. The system performing the process may be the system 100 of FIG. 1. For example, the steps of the process 900 can be performed by any of the impact analysis logic 124, impact analysis logic 164, and/or impact analysis application 160. The process 900 of FIG. 9 can be performed to provide a combined score based on a profile of an impact event.

The process 900 may begin by providing a user interface that displays virtual impact zones, the virtual impact zones corresponding to a plurality of physical impact zones on an impact apparatus (905). The system may also receive selection of a profile that identifies at least two target impact zones (910) via the user interface. The profile may include a series of target impact zones, the two target impact zones being identified in the series. The profile may include identifiers for target impact zones and non-target impact zones are those impact zones not identified in the profile. The profile may include weights for the impact zones of the impact apparatus, where a positive weight for an impact zone indicates a target impact zone and a negative weight for the impact zone indicates a non-target impact zone. In some implementations, a profile may include a primary target impact zone and a secondary target impact zone, the profile assigning a higher weight to a primary target impact zone than to a secondary target impact zone. In some implementations, a profile impact zones that are not target impact zones can have a zero weight.

The system may start a timer (915). The system may start the timer in response to a user command, e.g., an audible signal detected by the system. The system may start a timer in response to (or in conjunction with) a connected device, such as a piece of instrumented equipment A piece of instrumented equipment may be any sports equipment modified to send a start signal to the system. For example, instrumented equipment may be a ball that includes a motion detector (gyroscope, accelerometer, etc.) and is configured to send a start signal in response to a particular motion. In some implementations, the system may start a timer by recording a time of the start event.

The system may receive, from the impact apparatus, voltage information generated in response to an impact event (920). In some implementations, the voltage information is generated (in whole or in part) by impact analysis logic. The impact analysis logic may be included in a microcontroller of the impact apparatus. The impact analysis logic may be included in a computing device communicatively coupled to the impact apparatus. The voltage information can include the sampled voltage as described herein.

In some implementations, the voltage information is received in response to an impact event detected at an impact zone. In some implementations, the system may send a stop signal in response to the impact event, e.g., stopping a timer. Recording the time of the stop signal may be considered stopping the timer. The system may determine a response time for the impact event (925). This may be the difference between the recorded start time and stop time. The system may calculate (determine) a magnitude for the impact event attributable to each target impact zone (930). The magnitude may be calculated as discussed elsewhere, e.g., with regard to FIG. 5. The magnitude may be represented as a force. In some implementations, the system may calculate the magnitude of the impact event for each impact zone for which a detectable voltage was determined. Thus, for example, each impact zone, whether identified as a target impact zone or a non-target impact zone by the profile, may have a magnitude calculated representing the portion of the impact event attributable to the impact zone.

The system may determine a combined impact score for the impact event based on the magnitudes (935). The combined impact score can be calculated according to a formula associated with the profile. For example, some profiles may not be dependent on response time. A combined score which is independent of (not dependent on) response time may be calculated as Combined Score=ΣDesired Forces−ΣNon desired Forces, where a magnitude attributable to a target impact zone is a desired force and a magnitude attributable to a non-target impact zone is a non desired force. In some implementations, only desired forces are considered, e.g., Combined Score=ΣDesired Forces. Some profiles may be dependent on response time. In an implementation where the profile indicates a shorter response time is desired, the combined score may be calculated as

$$Combined Score = \frac{1}{Response} * ( \sum Desired Forces - \sum Non desired Forces ) .$$

Again, in some implementations, the non-desired forces may be removed (eliminated/ignored) from the calculation.

In some implementations one or more of the target impact zones can be weighted more than or less than other target impact zones and/or one or more non-target impact zones could be weighted more than or less than other non-target impact zones. In such an implementation the equation may be calculated as

$$Combined Score = \frac{1}{Response} * ( {\sum }_{i = 1}^{n} {w}_{i} {DPF}_{i} - {\sum }_{j = 1}^{m} {w}_{j} {NDF}_{j} ) ,$$

where n is the number of target impact zones (e.g., desired forces), m is the number of non-target zones (non-desired forces), and w is the weight assigned to each respective impact zone. In some implementations, the response time may also have a weight. This weight can also be customized, e.g., by a user of the application.

In some implementations, the system may be configured to determine session statistics based on the magnitude, response time, the combined score, etc. (940). The session statistics can include the total number (count) of impact events occurring during a session. The session statistics can include the average combined score time over the session. The session statistics can include the mean response time over the session, or other statistical operations (quartiles, etc.) applied to the response times in the session data. The session statistics may include the average (and/or mean, and/or quartile) of the combined score of the impact events during the session. As with FIG. 7, a session may be defined by the user. In some implementations, a session can be defined as a profile, e.g., so that a new profile defines a new session. In some implementations, session data can be associated with (defined by) a time period (e.g., all impacts for a certain profile performed within an hour, for a certain day, etc.) In some implementations, session data can further be associated with a user, e.g., a particular player. The player may be identified using automatic player detection. Detection of a new player may start a new session, or switch to a session associated with the newly detected player.

The system may update the user interface to display the combined score and the impact magnitudes (945). In some implementations, the system may also update the user interface with the response time for the impact event. In some implementations, updating the user interface includes changing an appearance of one or more of the impact zones in the user interface. In some implementations, updating the user interface may update session information. In some implementations, the user interface may include a graph for the session data, e.g., showing how response times, combined scores, and/or magnitude is trending. It is understood that steps 910 to 945 can be repeated several times, e.g., starting at 910, 915, or 920, depending on the implementation. In an implementation where the profile represents a series of target impact zones, the system may repeat steps 920 to 940 automatically and update the user interface 945 once the sequence has completed (e.g., impact events are received and analyzed for the series of target impact zones). In such implementations, a response time can also be calculated for the overall sequence.

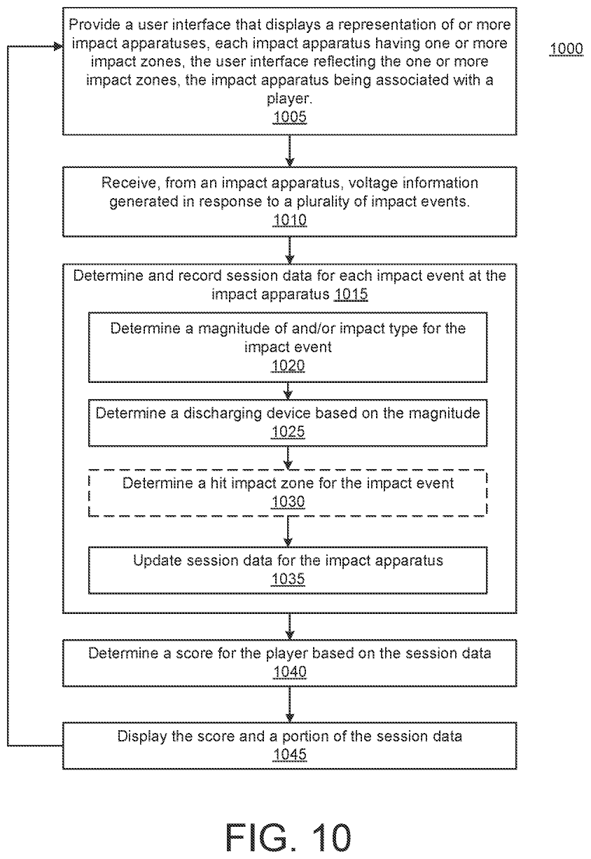

FIG. 10 is a flowchart that illustrates an example process 1000 for scoring repeated impacts to an impact apparatus, according to an implementation. The system performing the process may be the system 100 of FIG. 1. For example, the steps of the process 1000 can be performed by any of the impact analysis logic 124, impact analysis logic 164, and/or impact analysis application 160. The process 1000 of FIG. 10 can be performed to provide a response time and impact magnitude for an object impacting the impact apparatus.

The process 1000 may include providing a user interface that displays a representation of an impact apparatus. (1005). The display can include a representation of multiple impact apparatuses. Each impact apparatus may include an impact zone. For example, the impact apparatus may be padding worn by a player, e.g., a vest. In some implementations, the vest may be a single impact zone. In some implementations, the vest may include a plurality of impact zones. In some implementations, the user interface may be used to start a session. In some implementations, the system may be configured to start a new session in response to a voice command. During the session, the impact apparatus may receive a plurality of impact events. For example, the impact apparatus may be worn by a player during a simulated combat event, e.g., where players attempt to shoot other players using non-lethal projectiles (e.g., paintballs, chalk bullets, orbeez, airsoft BBs, or foam objects fired from a discharge device). Impacts on an impact apparatus may be tracked and scored during the session. Thus, the system receives voltage information for a plurality of impact events from the impact apparatus (1010) during the session. The system may determine and record session data for each impact event (1015). This session data is determined in real-time, e.g., as impact events occur.

Determining the session data can include determining a magnitude of the impact (1020). Some implementations may also determine a type of the impact. For example, the type may be a glancing impact or a direct impact. This may be determined by analysis of the voltage data over the impact period. As explained above with respect to calculating the magnitude of an impact event, the system may determine various features (integrals at various time periods during the impact event) of an impact event. These same features could be used as input to a classifier that determines whether the voltage data represents a direct impact or a glancing impact. In some implementations, the system may use a Fast Fourier Transfer to analyze the frequencies that comprise the voltage signal to determine properties of the projectile. In some implementations, a combined model may determine both the type of impact and the impact energy (magnitude) of the impact event. The system may use this information, e.g., the magnitude of the impact event and/or the magnitude and type of impact event, to determine a discharging device for the impact event (1025). In other words, the system may attribute the impact event to a type of discharging device. The system can do this where the different types (at least two different types) of discharging devices shoot the projectiles at different velocities and the projectiles all have a similar mass.

In implementations where the impact apparatus has more than one impact zone, the system may also determine which impact zone is the hit impact zone (1030). In such an implementation, different impact zones may be worth different points (e.g., may be weighted differently), so that impact events occurring on a first impact zone are weighted higher than impact events occurring on a second impact zone. The system updates the session data for the impact event for the impact apparatus (1035). This session data can include data from which to determine one or more of the following: number of impact events (i.e., the count of impact events) for the impact apparatus; the number of impact events attributed to each type of discharge device; for a particular discharge device: the number of direct impact events, the number of glancing impact events, the hit impact zones; for each hit impact zone: the number of direct impact events, the number of glancing impact events, the number of direct impact events by discharge device, the number of glancing impact events by discharge device.

The system may determine (calculate) a score for the impact apparatus (e.g., the player wearing the impact apparatus) based on the session statistics (1040). In some implementations, this score can be a running score calculated and displayed in real time. In some implementations, this score can be calculated after the session ends (e.g., after a predetermined time has elapsed from the session start time). In some implementations, scoring the session can be done according to a profile selected (e.g., a mode selected before the session). In such implementations, the profile may include weights for the discharge devices, the impact zones, the type of impact, etc. In some implementations, the weights may be fixed (not profile based). In some implementations the discharging devices may have the same weights. In some implementations, the discharging devices may have different weights. In some implementations different impact zones may have different weights. In some implementations, two or more (or all) of the impact zones may have the same weights. In some implementations, the impact types may have the same weights. In some implementations, the impact types may have different weights. In some implementations, no weights are used.

The score for an impact can be calculated based on a number of factors, including the type of discharging device, the impact zone(s) hit, the impact type, and weights (or lack of weights) assigned to these factors. In some implementations the weights may change depending on an expertise level. Some non-limiting examples follow, although implementations can include variations not expressly disclosed. In an implementation where the impact apparatus has n different discharge devices, the system may calculate the score according to: Score=Σᵢ₌₁ⁿwᵢcᵢ, where wᵢ is the weight assigned to the i^{th} discharge device and cᵢ is the number of times an impact event was attributed to that discharge device. In such implementations the type of impact (e.g., direct or glancing) may be used to determine a number of impacts attributed to the discharge device but may be weighted equally. In an implementation where the impact apparatus has n different discharge devices and m different impact zones, the score may be calculated as

$$Score = {\sum }_{j = 1}^{m} {s}_{j} {\sum }_{i = 1}^{n} {w}_{i} {c}_{i}$$

where sⱼ is the weight assigned to the impact zone j. In some implementations, the type of impact (e.g., direct vs. glancing) may be weighted differently, e.g., per device and/or per impact zone.

The system may display the score via the user interface (1045). In some implementations, the user interface may display some or all of the session data. In some implementations, the user interface may display the score for a plurality of impact apparatuses (e.g., all players in the combat simulation). Thus, the system may perform steps 1010 to 1040 for each impact apparatus included in the session. Process 1000 then ends, but may be repeated for another session.

In some aspects, the techniques described herein relate to a method including: receiving, from an impact apparatus, voltage information generated in response to a plurality of impact events, the impact apparatus including at least one impact zone configured to generate voltage in response to impact without a current producing device; for each impact event of the plurality of impact events: determining a discharging device attributable to the impact event based on at least one of a magnitude or an impact type determined from the voltage information, the discharging device being one of at least a first discharging device and a second discharging device, and updating session data for the impact apparatus, including updating a count of impacts attributable to the discharging device; calculating a score based on the session data; and providing a user interface displaying the score.

These and other aspects can include one or more of the following, alone or in combination. For example, the method may further include, for each of the plurality of impact events: determining a hit impact zone for the impact event, wherein the score is calculated based on the hit impact zone. As another example, the first discharging device may have a weight higher than the second discharging device. As another example, the impact apparatus has at least two impact zones and the session data includes, for each impact zone of the at least two impact zones, an impact zone count, the impact zone count reflecting a total number of impact events where the impact zone is a hit impact zone. In some such implementations, the impact zone count includes a number of impact events attributable to the first discharging device and a number of impact events attributable to the second discharging device. As another example, the impact type can be determined based on analysis of a voltage profile for the impact event. In some such examples, the impact type is one of a glancing impact and a direct impact and a glancing impact event has a lower weight in determining the score than a direct impact event.

In some aspects, the techniques described herein relate to a method including: providing a user interface that displays a plurality of virtual impact zones, the plurality of virtual impact zones corresponding to a plurality of physical impact zones on an impact apparatus, each impact zone of the plurality of physical impact zones configured to generate voltage in response to impact; receiving, via the user interface, selection of a target impact zone from the plurality of virtual impact zones; transmitting the target impact zone to the impact apparatus and recording a start time, wherein the impact apparatus changes an appearance of the target impact zone in response to receiving the target impact zone; receiving, from the impact apparatus, voltage information generated in response to an impact event, the impact event being an impact of an object on the impact apparatus; and in response to receiving the voltage information generated in response to the impact event: recording a stop time and calculating a response time based on elapsed time measured between the start time and the stop time; determining a magnitude of the impact event for the target impact zone based on the voltage information that is attributed to the target impact zone; calculating a score for the impact event based on a reciprocal of the response time and the magnitude; and updating the user interface to reflect the score.

These and other aspects can include one or more of the following, alone or in combination. For example, the voltage information can include, for at least one physical impact zone, voltage measured during an impact period lasting less than half a second. As another example, the target impact zone is a first target impact zone and the method can also include: receiving, via the user interface, selection of a second target impact zone of the plurality of virtual impact zones, wherein the first target impact zone is assigned a first weight and the second target impact zone is assigned a second weight; and transmitting the first target impact zone and the second target impact zone to the impact apparatus, wherein the impact apparatus further changes an appearance of the second target impact zone, wherein the changed appearance of the first target impact zone differs from the changed appearance of the second target impact zone. In some such example, in response to receiving the voltage information generated in response to the impact event the method can further include: determining a magnitude of the impact event for the second target impact zone based on the voltage information associated that is attributed to the second target impact zone; calculating a first weighted magnitude by applying the first weight to the magnitude of the impact event calculated for the first target impact zone; calculating a second weighted magnitude by applying the second weight to the magnitude of the impact event calculated for the second target impact zone; and calculating, as the score, a combined score for the impact event by combining the reciprocal of the response time with the first weighted magnitude and the second weighted magnitude. As another example, the start time is recorded in response to recognizing a voice command of a user. As another example, the start time is recorded in response to recognizing a predefined motion of a secondary sensor.

As another example, the method may further include: receiving, via the user interface, a sequence of target zones, the target impact zone being included in the sequence of target zones; and transmitting the sequence of target zones to the impact apparatus, wherein the impact apparatus is configured to serially change an appearance of the physical impact zones corresponding to the target impact zones in the sequence of target zones, with progression through the sequence of target zones being triggered by an impact event to any of the plurality of physical impact zones. In some such examples, the method may further include: receiving, from the impact apparatus, respective voltage information generated in response to each impact event; calculating a respective score for each target impact zone in the sequence of target zones from the voltage information attributed to the target impact zone; calculating a sequence score using the respective scores; and updating the user interface to reflect the sequence score.

As another example, receiving the target impact zone includes receiving an activity profile, the activity profile indicating target impact zones and non-target impact zones and calculating the score for the impact event can include: determining, for each impact zone, a magnitude of the impact event based on voltage information attributable to the impact zone; calculating a target magnitude by combining the magnitudes for the target impact zones; calculating a non-target magnitude by combining the magnitudes for the non-target impact zones; and calculating the score as a difference between the target magnitude and the non-target magnitude combined with the reciprocal of the response time. In some such examples, the activity profile includes a respective weight for each impact zone and wherein, for each impact zone, the magnitude of the impact event for the impact zone is multiplied by the respective weight for the impact zone. Calculating the score as a difference between the target magnitude and the non-target magnitude can be accomplished by using negative weights for non-target zones. In some examples, the weights correspond to an expertise level. In some examples, at least one physical impact zone is in padding worn by a user striking the impact apparatus. In some examples, the method further includes: updating the user interface to display the magnitude of the impact for each impact zone.

In some aspects, the techniques described herein relate to a method including: providing a user interface that displays a plurality of virtual impact zones, the plurality of virtual impact zones displayed in the user interface corresponding to a plurality of physical impact zones on an impact apparatus, each impact zone of the plurality of physical impact zones configured to generate voltage in response to impact; receiving selection of an object, the object having a known mass; receiving, from the impact apparatus, voltage information generated in response to an impact event, the impact event being an impact of the object on the impact apparatus; and in response to receiving the voltage information generated in response to the impact event: determining a hit impact zone from the voltage information; determining a velocity of the object from the voltage information and the known mass; and updating the user interface to identify the hit impact zone and to display the velocity.

These and other aspects can include one or more of the following, alone or in combination. For example, updating the user interface can occur in real-time. As another example, the method can further include: receiving selection of a target impact zone of the plurality of virtual impact zones; and in response to receiving the voltage information generated in response to the impact event: determining whether the target impact zone matches the hit impact zone, and updating the user interface with an indication of whether the target impact zone matches the hit impact zone. In some examples, the method may further include: transmitting the hit impact zone to the impact apparatus; and changing an appearance of the hit impact zone at the impact. As another example, in response to receiving the voltage information generated in response to the impact, the method may further include updating a session record stored in a memory, updating the session record including adding the velocity of the object to the session record and updating an impact event count in the session record; calculating an average velocity based on the session record; and updating the user interface to display the average velocity. In some examples, the method further includes receiving an instruction to start a new session; and initializing the session record. The instruction to start the new session may result from selection of a new object with a different known mass.

As another example, the method may also include: receiving selection of a target impact zone of the plurality of virtual impact zones; and in response to receiving the voltage information generated in response to the impact: updating a session record stored in memory, updating the session record including adding the velocity of the object to the session record, updating an impact event count in the session record, and recording a determination of whether the target impact zone matches the hit impact zone in the session record, calculating an average velocity based on the session record, calculating a hit ratio based on the session record, and updating the user interface to display the average velocity, the hit ratio, and the impact event count. The method can also include updating the user interface to display an indication of whether the target impact zone matches the hit impact zone.

As another example, he plurality of physical impact zones can be arranged in three columns with one impact zone of the plurality of physical impact zones surrounding the three columns. In some examples, at least two columns of the three columns each include three physical impact zones.

As another example, determining the velocity of the object from the voltage information and the known mass includes: determining a peak voltage over an impact period at the hit impact zone; determining an impact energy Et from the peak voltage based on calibration data, wherein the impact energy Et has a direct relationship with the peak voltage; and calculating the velocity according to

$$v = \sqrt{2 \frac{{E}_{t}}{m}}$$

where m is the known mass.

In some aspects, the techniques described herein relate to a method including: providing a user interface that displays a plurality of virtual impact zones, the plurality of virtual impact zones displayed in the user interface corresponding to a plurality of physical impact zones on an impact apparatus, each impact zone of the plurality of physical impact zones configured to generate voltage in response to impact; receiving, via the user interface, a profile, the profile including identification of at least two impact zones of the plurality of physical impact zones as target impact zones, remaining impact zones in the plurality of physical impact zones being non-target impact zones; receiving, from the impact apparatus, voltage information generated in response to an impact event, the impact event being an impact of an object on the impact apparatus; and in response to receiving the voltage information generated in response to the impact event: determining, for each target impact zone, a magnitude of the impact event for the target impact zone based on the voltage information associated with the target impact zone; calculating a score for the impact event based the magnitudes; and updating the user interface to reflect the score.

These and other aspects can include one or more of the following, alone or in combination. For example, receiving the target impact zone includes receiving an activity profile, the activity profile indicating target impact zones and non-target impact zones and wherein calculating the score for the impact event includes: determining, for each impact zone, a magnitude of the impact event based on voltage information attributable to the impact zone; calculating a target magnitude by combining the magnitudes for the target impact zones; calculating a non-target magnitude by combining the magnitudes for the non-target impact zones; and calculating the score as a difference between the target magnitude and the non-target magnitude.

As another example, the activity profile includes a respective weight for each impact zone and wherein, for each impact zone, the magnitude of the impact event for the impact zone is multiplied by the respective weight for the impact zone. In some such examples, calculating the score as a difference between the target magnitude and the non-target magnitude is accomplished by using negative weights for non-target zones. In some examples, the weights may correspond to an expertise level. In some examples, at least one physical impact zone is in padding worn by a user striking the impact apparatus. In some examples, updating the user interface to display the magnitude of the impact for each impact zone.

As another example. the method may further include: recording a start time in response to a command from a user; and in response to receiving the voltage information: recording a stop time, and calculating a response time based on elapsed time measured by the start time and the stop time, wherein calculating the score is further based on a reciprocal of the response time.

In some aspects, the techniques described herein relate to a system including: an impact apparatus with an impact zone configured to generate a voltage in response to an impact of an object; at least one processor; and memory storing instructions that, when executed by the at least one processor, cause the system to perform the method of any preceding claim.

Implementations of the various techniques described herein may be implemented in digital electronic circuitry, or in computer hardware, firmware, software, or in combinations of them. Implementations may implemented as a computer program product, i.e., a computer program tangibly embodied in an information carrier, e.g., in a non-transitory machine-readable storage device (computer-readable medium) for processing by, or to control the operation of, data processing apparatus, e.g., a programmable processor, a computer, or multiple computers. A computer program, such as the computer program(s) described above, can be written in any form of programming language, including compiled or interpreted languages, and can be deployed in any form, including as a stand-alone program or as a module, component, subroutine, or other unit suitable for use in a computing environment. A computer program can be deployed to be processed on one computer or on multiple computers at one site or distributed across multiple sites and interconnected by a communication network.

Many of the method steps may be performed by one or more programmable processors executing a computer program to perform functions by operating on input data and generating output. Method steps also may be performed by, and an apparatus may be implemented as, special purpose logic circuitry, e.g., an FPGA (field programmable gate array) or an ASIC (application-specific integrated circuit).

Processors suitable for the processing of a computer program include, by way of example, both general and special purpose microprocessors, and any one or more processors formed in a substrate of any kind of digital computer. Generally, a processor will receive instructions and data from a read-only memory or a random access memory or both. Elements of a computer may include at least one processor for executing instructions and one or more memory devices for storing instructions and data. Generally, a computer also may include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory may be supplemented by, or incorporated in, special purpose logic circuitry.

To provide for interaction with a user, implementations may be implemented on a computer having a display device, e.g., a touch screen, a monitor, a projection, etc. for displaying information to the user and an input device, e.g., keyboard, a pointing device, e.g., a finger, a stylus, a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Implementations may be implemented in a computing system that includes a back-end component, e.g., as a data server, or that includes a middleware component, e.g., an application server, or that includes a front-end component, e.g., a client computer having a graphical user interface or a Web browser through which a user can interact with an implementation, or any combination of such back-end, middleware, or front-end components. Components may be interconnected by any form or medium of digital data communication, e.g., a communication network. Examples of communication networks include a local area network (LAN) and a wide area network (WAN), e.g., the Internet.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments. It should be understood that they have been presented by way of example only, not limitation, and various changes in form and details may be made. Any portion of the apparatus and/or methods described herein may be combined in any combination, except mutually exclusive combinations. The embodiments described herein can include various combinations and/or sub-combinations of the functions, components and/or features of the different embodiments described.

1. A method comprising:
receiving, from an impact apparatus, voltage information generated in response to a plurality of impact events, the impact apparatus including at least one impact zone configured to generate voltage in response to impact without a current producing device;
for each impact event of the plurality of impact events:
determining a discharging device attributable to the impact event based on at least one of a magnitude or an impact type determined from the voltage information, the discharging device being one of at least a first discharging device and a second discharging device, and
updating session data for the impact apparatus, including updating a count of impacts attributable to the discharging device;
calculating a score based on the session data; and
providing a user interface displaying the score.

2. The method of claim 1, further comprising, for each of the plurality of impact events:
determining a hit impact zone for the impact event,
wherein the score is calculated based on the hit impact zone.

3. The method of claim 1, wherein the first discharging device has a weight higher than the second discharging device.

4. The method of claim 1, wherein the impact apparatus has at least two impact zones and the session data includes, for each impact zone of the at least two impact zones, an impact zone count, the impact zone count reflecting a total number of impact events where the impact zone is a hit impact zone.

5. The method of claim 4, wherein the impact zone count includes a number of impact events attributable to the first discharging device and a number of impact events attributable to the second discharging device.

6. The method of claim 1, wherein the impact type is determined based on analysis of a voltage profile for the impact event.

7. The method of claim 6, wherein the impact type is one of a glancing impact and a direct impact and a glancing impact event has a lower weight in determining the score than a direct impact event.

8. A method comprising:
providing a user interface that displays a plurality of virtual impact zones, the plurality of virtual impact zones corresponding to a plurality of physical impact zones on an impact apparatus, each impact zone of the plurality of physical impact zones configured to generate voltage in response to impact;
receiving, via the user interface, selection of a target impact zone from the plurality of virtual impact zones;
transmitting the target impact zone to the impact apparatus and recording a start time, wherein the impact apparatus changes an appearance of the target impact zone in response to receiving the target impact zone;
receiving, from the impact apparatus, voltage information generated in response to an impact event, the impact event being an impact of an object on the impact apparatus; and
in response to receiving the voltage information generated in response to the impact event:
recording a stop time and calculating a response time based on elapsed time measured between the start time and the stop time;
determining a magnitude of the impact event for the target impact zone based on the voltage information that is attributed to the target impact zone;
calculating a score for the impact event based on a reciprocal of the response time and the magnitude; and
updating the user interface to reflect the score.

9. The method of claim 8, wherein the voltage information includes, for at least one physical impact zone, voltage measured during an impact period lasting less than half a second.

10. The method of claim 8, wherein the target impact zone is a first target impact zone and the method includes:
receiving, via the user interface, selection of a second target impact zone of the plurality of virtual impact zones, wherein the first target impact zone is assigned a first weight and the second target impact zone is assigned a second weight; and
transmitting the first target impact zone and the second target impact zone to the impact apparatus, wherein the impact apparatus further changes an appearance of the second target impact zone, wherein the changed appearance of the first target impact zone differs from the changed appearance of the second target impact zone.

11. The method of claim 10, wherein in response to receiving the voltage information generated in response to the impact event the method further comprises:
determining a magnitude of the impact event for the second target impact zone based on the voltage information associated that is attributed to the second target impact zone;
calculating a first weighted magnitude by applying the first weight to the magnitude of the impact event calculated for the first target impact zone;
calculating a second weighted magnitude by applying the second weight to the magnitude of the impact event calculated for the second target impact zone; and
calculating, as the score, a combined score for the impact event by combining the reciprocal of the response time with the first weighted magnitude and the second weighted magnitude.

12. The method of claim 8, further comprising:
receiving, via the user interface, a sequence of target zones, the target impact zone being included in the sequence of target zones; and
transmitting the sequence of target zones to the impact apparatus, wherein the impact apparatus is configured to serially change an appearance of the physical impact zones corresponding to the target impact zones in the sequence of target zones, with progression through the sequence of target zones being triggered by an impact event to any of the plurality of physical impact zones.

13. The method of claim 12, further comprising:
receiving, from the impact apparatus, respective voltage information generated in response to each impact event;
calculating a respective score for each target impact zone in the sequence of target zones from the voltage information attributed to the target impact zone;
calculating a sequence score using the respective scores; and
updating the user interface to reflect the sequence score.

14. The method of claim 8, wherein receiving the target impact zone includes receiving an activity profile, the activity profile indicating target impact zones and non-target impact zones and wherein calculating the score for the impact event includes:
determining, for each impact zone, a magnitude of the impact event based on voltage information attributable to the impact zone;
calculating a target magnitude by combining the magnitudes for the target impact zones;
calculating a non-target magnitude by combining the magnitudes for the non-target impact zones; and
calculating the score as a difference between the target magnitude and the non-target magnitude combined with the reciprocal of the response time.

15. The method of claim 14, wherein the activity profile includes a respective weight for each impact zone and wherein, for each impact zone, the magnitude of the impact event for the impact zone is multiplied by the respective weight for the impact zone.

16. The method of claim 15, wherein calculating the score as a difference between the target magnitude and the non-target magnitude is accomplished by using negative weights for non-target zones.

17. The method of claim 15, wherein the weights correspond to an expertise level.

18. The method of claim 14, wherein at least one physical impact zone is in padding worn by a user striking the impact apparatus.

19. The method of claim 14, further comprising:
updating the user interface to display the magnitude of the impact for each impact zone.

20. The method of claim 8, wherein the start time is recorded in response to recognizing a voice command of a user.

21. The method of claim 8, wherein the start time is recorded in response to recognizing a predefined motion of a secondary sensor.

22.-42. (canceled)
